(19) United States
(12) Patent Application Publication
(10) Pub. No.: US 2024/0285657 A1
(43) Pub. Date: Aug. 29, 2024

(54) VETERINARY FORMULATION OF METRONIDAZOLE, AND RELATED METHODS

(71) Applicant: Verte Therapeutics, LLC, New York, NY (US)

(72) Inventors: Lesley Corene Rausch-Derra, Parkville, MO (US); Susan Mancini Cady, Yardley, PA (US); Rex Allen Henry, Lenexa, KS (US); Robbin Lynn Koenig, Overland Park, KS (US); Michael Radomsky, Chagrin Falls, OH (US); Donald Duwe, Strongsville, OH (US); Jessica Williams, Broadview Heights, OH (US); Arthur Tipton, Birmingham, AL (US)

(21) Appl. No.: 18/424,367

(22) Filed: Jan. 26, 2024

Related U.S. Application Data
(60) Provisional application No. 63/448,810, filed on Feb. 28, 2023.

(51) Int. Cl. A61K 31/7024; A61K 31/22; A61K 31/4164; A61K 47/14; A61P 33/02

(52) U.S. Cl. CPC A61K 31/7024; A61K 31/22; A61K 31/4164; A61K 47/14; A61P 33/02

(57) ABSTRACT

Disclosed is a pharmaceutical composition, comprising metronidazole or a pharmaceutically acceptable salt thereof, sucrose acetate isobutyrate (SAIB), and ethyl acetate. Also disclosed is a method treating or preventing a protozoal or bacterial infection or disease, comprising administering to a non-human subject in need thereof a therapeutically effective amount of the pharmaceutical composition.

### RELATED APPLICATION

This application claims benefit of priority to U.S. Provisional Patent Application No. 63/448,810, filed Feb. 28, 2023.

### BACKGROUND

Protozoal infections, are common parasitic infections in dogs and cats. For example, Giardiasis can cause diarrhea in dogs. It is caused by an intestinal parasite called Giardia, which can be found in feces-contaminated soil, food, and water. The lifecycle of Giardia is composed of two stages. The mature parasites, or trophozoites, live in the small intestine where they multiply and eventually become cysts. Cysts are the infective stage and are shed into the feces of the infected animal. They can survive for several weeks in the environment as cysts, and when they are ingested by an unsuspecting host, they turn into trophozoites and repeat the life cycle. Symptoms of infection may include diarrhea, weight loss, failure to gain weight, vomiting, dehydration, and poor coat appearance.

Clinically recognized cases are often treated with metronidazole. This treatment requires a relatively long period of administration (5-10 days), which may result in a low compliance rate. Available oral forms are inconvenient for administration to small animals. Oral forms of metronidazole are bitter tasting and can be difficult to dose in animals. Accordingly, formulations for more convenient administration for protozoal or bacterial infections are needed.

### SUMMARY OF THE INVENTION

The present invention provides a pharmaceutical composition, comprising metronidazole or a pharmaceutically acceptable salt thereof; sucrose acetate isobutyrate (SAIB); and ethyl acetate.

Also disclosed is a method of delivering metronidazole or a pharmaceutically acceptable salt thereof, comprising administering to a non-human subject in need thereof an effective amount of any one of the pharmaceutical compositions disclosed herein, thereby delivering the metronidazole or a pharmaceutically acceptable salt thereof.

Also disclosed is a method of treating or preventing a protozoal or bacterial infection or disease, comprising administering to a non-human subject in need thereof a therapeutically effective amount of any one of the pharmaceutical compositions disclosed herein, thereby treating or preventing the protozoal or bacterial infection or disease.

### BRIEF DESCRIPTION OF THE DRAWINGS

FIG. 1 displays individual animal syringe weights.

FIG. 2 displays injection site volumes by group.

FIG. 3A shows plasma metronidazole concentrations for individual dogs of Group 1 (nominal % concentration of metronidazole shown).

FIG. 3B shows plasma metronidazole concentrations for individual dogs of Group 2 (nominal % concentration of metronidazole shown).

FIG. 3C shows plasma metronidazole concentrations for individual dogs of Group 3 (nominal % concentration of metronidazole shown).

FIG. 3D shows plasma metronidazole concentrations for individual dogs of Group 4 (nominal % concentration of metronidazole shown).

FIG. 3E shows plasma metronidazole concentrations for individual dogs of Group 5 (nominal % concentration of metronidazole shown).

FIG. 3F shows plasma metronidazole concentrations for individual dogs of Group 6 (nominal % concentration of metronidazole shown).

FIG. 4A shows concentration versus time curves for individual dogs receiving Formula 1 (actual % concentration of metronidazole shown).

FIG. 4B shows concentration versus time curves for individual dogs receiving Formula 2 (actual % concentration of metronidazole shown).

FIG. 5A shows concentration versus time curves for individual dogs in Group A (actual % concentration of metronidazole shown).

FIG. 5B shows concentration versus time curves for individual dogs in Group B (actual % concentration of metronidazole shown).

FIG. 5C shows concentration versus time curves for individual dogs in Group C (actual % concentration of metronidazole shown).

FIG. 5D shows concentration versus time curves for individual dogs in Group D (actual % concentration of metronidazole shown).

FIG. 6 displays a plot of daily FCC of individual animals. ND: non-detect; <67 CPG. The Dog ID was inset at the bottom left corner of each cell.

FIG. 7 displays percent treatment success and mean percent fecal reduction according to treatment group.

FIG. 8A shows plasma metronidazole concentrations for individual dogs following administration of formulation 8A.

FIG. 8B shows plasma metronidazole concentrations for individual dogs following administration of formulation 8B.

FIG. 8C shows plasma metronidazole concentrations for individual dogs following administration of formulation 8C.

FIG. 8D shows plasma metronidazole concentrations for individual dogs following administration of formulation 8D.

FIG. 8E shows plasma metronidazole concentrations for individual dogs following administration of formulation 8E.

FIG. 8F shows plasma metronidazole concentrations for individual dogs following administration of formulation 8F.

FIG. 9 shows the % Fecal Cyst Reduction and % Trophozoite Reduction following treatment with 125 or 150 mg/kg metronidazole (75:25 SAIB:EA) with 36% (nominal) metronidazole.

### DETAILED DESCRIPTION OF THE INVENTION

The present invention provides compositions comprising metronidazole or a pharmaceutically acceptable salt thereof, sucrose acetate isobutyrate (SAIB); and ethyl acetate. The compositions are formulated as an injectable for extended-release.

The compositions are formulated as a depot injectable formulation to treat veterinary parasitic infections, e.g. giardia infections in dogs and cats.

Upon injection of the formulation, the ethyl acetate dissipates into the surrounding tissues leaving behind the SAIB/drug depot which releases the drug by diffusion from the SAIB. The SAIB gradually degrades in vivo by enzymatic hydrolysis into sucrose and its aliphatic acids. The extended-release formulation is designed to slowly release the drug over a period of time, e.g. 5 to 7 days.

### Compositions of the Invention

The present invention provides a pharmaceutical composition, comprising
(i) metronidazole or a pharmaceutically acceptable salt thereof;

(ii) sucrose acetate isobutyrate (SAIB); and
(iii) ethyl acetate.

In certain embodiments, the pharmaceutical composition comprising about 20% to about 50% w/w metronidazole or a pharmaceutically acceptable salt thereof.

In certain embodiments, the pharmaceutical composition comprising about 20% to about 45% w/w metronidazole or a pharmaceutically acceptable salt thereof.

In certain embodiments, the pharmaceutical composition comprising about 30% to about 45% w/w metronidazole or a pharmaceutically acceptable salt thereof.

In certain embodiments, the pharmaceutical composition comprising about 20% to about 40% w/w metronidazole or a pharmaceutically acceptable salt thereof.

In certain embodiments, the pharmaceutical composition comprising about 30% to about 40% w/w metronidazole or a pharmaceutically acceptable salt thereof.

In certain embodiments, the pharmaceutical composition comprising about 34% to about 36% w/w metronidazole or a pharmaceutically acceptable salt thereof.

In certain embodiments, the pharmaceutical composition comprising about 36% to about 44% w/w metronidazole or a pharmaceutically acceptable salt thereof.

In certain embodiments, the pharmaceutical composition comprising about 38% to about 42% w/w metronidazole or a pharmaceutically acceptable salt thereof.

In certain embodiments, the pharmaceutical composition, comprising about 30% to about 70% w/w SAIB.

In certain embodiments, the pharmaceutical composition comprising about 40% to about 60% w/w SAIB.

In certain embodiments, the pharmaceutical composition comprising about 50% to about 60% w/w SAIB.

In certain embodiments, the pharmaceutical composition comprising about 50% to about 55% w/w SAIB.

In certain embodiments, the pharmaceutical composition comprising about 5% to about 20% w/w ethyl acetate.

In certain embodiments, the pharmaceutical composition comprising about 10% to about 20% w/w ethyl acetate.

In certain embodiments, the pharmaceutical composition comprising about 10% to about 15% w/w ethyl acetate.

In certain embodiments, the pharmaceutical composition further comprising (iv) an excipient.

In certain embodiments, the excipient comprises medium-chain triglycerides.

In certain embodiments, the excipient comprises C8 and C10 triglycerides.

In certain embodiments, the pharmaceutical composition comprising about 5% to about 15% w/w excipient.

In certain embodiments, the pharmaceutical composition comprising about 5% to about 10% w/w excipient.

In certain embodiments, the pharmaceutical composition comprising about 8% to about 12% w/w excipient.

In certain embodiments, the weight ratio of SAIB to ethyl acetate is about 60:40 to about 90:10.

In certain embodiments, the weight ratio of SAIB to ethyl acetate is about 70:30 to about 85:15.

In certain embodiments, the weight ratio of SAIB to ethyl acetate is about 75:25 to about 80:20.

In certain embodiments, the weight ratio of SAIB to ethyl acetate is about 75:25.

In certain embodiments, the weight ratio of SAIB to ethyl acetate is about 80:20.

In certain embodiments, the metronidazole or salt thereof has a particle size distribution (D₉₀) range of about 30 μm to about 300 μm.

In certain embodiments, the metronidazole or salt thereof has a particle size distribution (D₉₀) range of about 40 μm to about 100 μm.

In certain embodiments, the metronidazole or salt thereof has a particle size distribution (D₉₀) range of about 30 μm to about 100 μm.

In certain embodiments, the metronidazole or salt thereof has a particle size distribution (D₉₀) range of about 100 μm to about 300 μm.

In certain embodiments, the metronidazole or salt thereof has a particle size distribution (D₉₀) range of about 145 μm to about 225 μm.

In certain embodiments, the metronidazole or salt thereof has a particle size distribution (D₉₀) range of about 30 μm to about 40 μm.

In certain embodiments, the metronidazole or salt thereof has a particle size distribution (D₉₀) range of about 100 μm to about 225 μm.

In certain embodiments, the metronidazole or salt thereof has a particle size distribution (D₉₀) range of about 100 μm to about 125 μm.

In certain embodiments, the metronidazole or salt thereof has a particle size distribution (D₅₀) range of about 10 μm to about 120 μm.

In certain embodiments, the metronidazole or salt thereof has a particle size distribution (D₅₀) range of about 20 μm to about 110 μm.

In certain embodiments, the metronidazole or salt thereof has a particle size distribution (D₅₀) range of about 25 μm to about 100 μm.

In certain embodiments, the metronidazole or salt thereof has a particle size distribution (D₅₀) range of about 15 μm to about 35 μm.

In certain embodiments, the metronidazole or salt thereof has a particle size distribution (D₅₀) range of about 90 μm to about 110 μm.

In certain embodiments, the metronidazole or salt thereof has a particle size distribution (D₁₀) range of about 3 μm to about 40 μm.

In certain embodiments, the metronidazole or salt thereof has a particle size distribution (D₁₀) range of about 4 μm to about 37 μm.

In certain embodiments, the metronidazole or salt thereof has a particle size distribution (D₁₀) range of about 3 μm to about 10 μm.

In certain embodiments, the metronidazole or salt thereof has a particle size distribution (D₁₀) range of about 30 μm to about 40 μm.

In certain embodiments, the pharmaceutical composition comprising.
(i) about 30% to about 36% w/w metronidazole or a pharmaceutically acceptable salt thereof:
(ii) about 48% to about 52.5% w/w SAIB; and
(iii) about 16% to about 17.5% w/w ethyl acetate.

In certain embodiments, the pharmaceutical composition comprising.
(i) about 30% w/w metronidazole or a pharmaceutically acceptable salt thereof:
(ii) about 52.5% w/w SAIB; and
(iii) about 17.5% w/w ethyl acetate.

In certain embodiments, the pharmaceutical composition comprising.
(i) about 36% w/w metronidazole or a pharmaceutically acceptable salt thereof:
(ii) about 48% w/w SAIB; and
(iii) about 16% w/w ethyl acetate.

In certain embodiments, the pharmaceutical composition is an extended-release composition.

In certain embodiments, the pharmaceutical composition is subject to a sterilization process.

In certain embodiments, the pharmaceutical composition is gamma irradiated. In certain embodiments, the pharmaceutical composition is gamma irradiated at about 10 to about 30 kGy. In certain embodiments, the pharmaceutical composition is gamma irradiated at about 10 to about 15 kGy. In other embodiments, the pharmaceutical composition is gamma irradiated at about 10 kGy.

In certain embodiments, the pharmaceutical composition is cooled with dry ice during the sterilization process. In other embodiments, the pharmaceutical composition is at room temperature during the sterilization process.

### Methods of the Invention

The present invention provides a method of delivering metronidazole or a pharmaceutically acceptable salt thereof, comprising administering to a non-human subject in need thereof an effective amount of any one of the pharmaceutical compositions disclosed herein, thereby delivering the metronidazole or a pharmaceutically acceptable salt thereof.

In certain embodiments, the non-human subject is a canine.

In certain embodiments, the canine is a puppy.

In certain embodiments, the non-human subject is a feline.

In certain embodiments, the feline is a kitten.

In certain embodiments, the pharmaceutical composition is administered by injection.

In certain embodiments, the pharmaceutical composition is administered by subcutaneous injection.

The present invention provides a method of treating or preventing a protozoal or bacterial infection or disease, comprising administering to a non-human subject in need thereof a therapeutically effective amount of any one of the pharmaceutical compositions disclosed herein, thereby treating or preventing the protozoal or bacterial infection or disease.

In certain embodiments, the method for treating or preventing a protozoal infection or disease in the non-human subject.

In certain embodiments, the protozoal infection or disease is caused by Giardia spp., Trichomonas spp., Entamoeba spp., or Balantidium spp.

In certain embodiments, the protozoal infection or disease is caused by Giardia duodenalis or Entamoeba histolytica.

In certain embodiments, the protozoal disease is giardiasis, trichomoniasis, amoebiasis or balantidiasis.

In certain embodiments, the method for treating or preventing a bacterial infection or disease in the non-human subject.

In certain embodiments, the bacterial infection or disease is an anaerobic bacterial infection or disease.

In certain embodiments, the bacterial infection or disease is caused by anaerobic bacteria.

In certain embodiments, the bacterial infection or disease is caused by Clostridium spp.

In certain embodiments, the bacterial infection or disease is caused by enteric bacteria.

In certain embodiments, the protozoal or bacterial infection or disease occurs in the gastrointestinal tract of the non-human subject.

In certain embodiments, the method for treating the protozoal or bacterial infection or disease in the non-human subject.

In certain embodiments, the method for preventing the protozoal or bacterial infection or disease in the non-human subject.

In certain embodiments, the pharmaceutical composition is administered to a preoperative subject. In certain embodiments, the pharmaceutical composition is administered to a preoperative subject as a prophylaxis.

In certain embodiments, the pharmaceutical composition is administered to a postoperative subject.

In certain embodiments, the pharmaceutical composition alleviates one or more symptoms in the non-human subject.

In certain embodiments, the one or more symptoms are selected from the group consisting of diarrhea, gas, abdominal discomfort, nausea, and vomiting.

In certain embodiments, the non-human subject is a canine.

In certain embodiments, the canine is a puppy.

In certain embodiments, the non-human subject is a feline.

In certain embodiments, the feline is a kitten.

In certain embodiments, the pharmaceutical composition is administered by injection.

In certain embodiments, the pharmaceutical composition is administered by subcutaneous injection.

In certain embodiments, a dosage of about 50 mg/kg to about 200 mg/kg metronidazole or a pharmaceutically acceptable salt thereof is administered to the non-human subject.

In certain embodiments, the dosage is about 75 mg/kg. In other embodiments, dosage is about 100 mg/kg. In other embodiments, the dosage is about 125 mg/kg. In other embodiments, in the dosage is about 150 mg/kg. In other embodiments, the dosage is about 175 mg/kg.

In certain embodiments, the dosage is administered in a single dose.

In certain embodiments, the method is for treating or preventing a protozoal infection or disease. In other embodiments, the method is for treating or preventing a protozoal infection. In certain embodiments, the method is for treating or preventing a protozoal disease.

In certain embodiments, the method is for treating or preventing a bacterial infection or disease. In other embodiments, the method is for treating or preventing a bacterial infection. In other embodiments, the method is for treating or preventing a bacterial disease.

In certain embodiments, the method is for treating a protozoal infection or disease. In other embodiments, the method is for treating a protozoal infection. In other embodiments, the method is for treating a protozoal disease.

In certain embodiments, the method is for treating a bacterial infection or disease. In other embodiments, the method is for treating a bacterial infection. In other embodiments, the method is for treating a bacterial disease.

In certain embodiments, the method is for preventing a protozoal infection or disease. In other embodiments, the method is for preventing a protozoal infection. In other embodiments, the method is for preventing a protozoal disease.

In certain embodiments, the method is for preventing a bacterial infection or disease. In other embodiments, the method is for preventing a bacterial infection. In other embodiments, the method is for preventing a bacterial disease.

### Administration

The formulations used in the invention may be administered in pharmaceutically acceptable solutions, suspensions, pastes, or gels, each of which may routinely contain pharmaceutically acceptable concentrations of salt, buffering agents, preservatives, compatible carriers, adjuvants, and optionally other therapeutic ingredients.

For use in therapy, an effective amount of the composition of the invention can be administered to a subject by any mode that delivers the compound or composition to the desired location or surface. Administering the pharmaceutical composition of the present invention may be accomplished by any means known to the skilled artisan. Routes of administration include but are not limited to oral, intravenous, intramuscular, intraperitoneal, subcutaneous, direct injection (for example, into a tumor or abscess), mucosal, inhalation, and topical.

In certain embodiments, the composition is administered systemically. In certain preferred embodiments, the composition is administered intravenously.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

The pharmaceutical composition of the present invention is administered by injection, e.g. subcutaneous injection. Upon injection of the formulation, the ethyl acetate dissipates into the surrounding tissues leaving behind the SAIB/drug depot which releases the drug by diffusion from the SAIB. The SAIB gradually degrades in-vivo by enzymatic hydrolysis into sucrose and its aliphatic acids. The gradual degradation results in a slowly release of the metronidazole over 5-7 days.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an optional added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

In addition to the formulations described above, the compounds may also be formulated as a depot preparation. Such long-acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended-release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 3 days, preferably at least 5 days, and more preferably 30-60 days. Long-term sustained release implants are well-known to those of ordinary skill in the art and include some of the release systems described above.

### Definitions

Metronidazole is a nitroimidazole compound used to treat trichomoniasis, amebiasis, inflammatory lesions of rosacea, and bacterial infections, as well as prevent postoperative infections. Metronidazole has the following chemical structure:

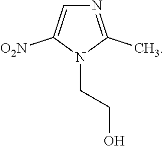 [SMILES: Cc1ncc([N+](=O)[O-])n1CCO]

The terms “treat” and “treating” as used herein refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease.

A “subject” or “patient” as used herein refers to a living mammal. In various embodiments, a patient is a non-human mammal, including, without limitation, a mouse, rat, hamster, guinea pig, rabbit, sheep, goat, cat, dog, pig, horse, cow, or non-human primate. In certain embodiments a patient is a human. A “non-human subject” or “non-human patient” as used herein excludes humans.

“Effective amount” as used herein refers to any amount that is sufficient to achieve a desired biological effect.

“Therapeutically effective amount” as used herein refers to any amount that is sufficient to achieve a desired therapeutic effect, e.g., treating the infection or disease.

“Active ingredient”, “therapeutically active ingredient”, “active agent”, “drug” or “drug substance” as used herein means the active ingredient of a pharmaceutical, also known as an active pharmaceutical ingredient (API).

“Amorphous” as used herein refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid-like properties occurs at a “glass transition”, typically defined as a second-order phase transition.

“Crystalline” as used herein refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically a first-order phase transition (“melting point”). In the context of the present invention, a crystalline active ingredient means an active ingredient with crystallinity of greater than 75%. In certain embodiments the crystallinity is suitably greater than 90%. In other embodiments, the crystallinity is greater than 95%. In other embodiments, the crystallinity is less than 10%, or less than 5%.

“Drug Loading” as used herein refers to the percentage of active ingredient(s) on a mass basis in the total mass of the formulation.

“Particle Size Distribution” or “D_{X}” as used herein means the point on the particle size distribution curve below which X % of the given particles fall. For example, a D₁₀ of 10 microns means 10% of the particles have a particles diameter of 10 microns or less.

The term “about” refers to variations in numerical values typically encountered by one of skill in the art of respirable formulations, including variations of plus or minus 0.1%, 0.5%, 1%, 2%, 3%, 4%, 5%, 6%, 7%, 8%, 9%, or 10% of a numerical value described herein.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

Unless otherwise stated, or clear from the context, numerical ranges include both the endpoints and any value between them.

### Examples

Having now described the present invention in detail, the same will be more clearly understood by reference to the following examples, which are included herewith for purposes of illustration only and are not intended to be limiting of the invention.

### Example 1: Preparation of Compositions

Compositions of the present invention were prepared by the following method. The Silverson Mixer Homogenizer was used to manufacture the formulation for improved mixing and physical characteristics of the formulation, such as flowability and syringeability.
1) SAIB was heated to 60-70° C. to reduce viscosity (SAIB is highly viscous at RT);
2) The SAIB was transferred to a mixing vessel and solvent (ethyl acetate) added;
3) The solution was mixed for 1 hour to obtain uniform solution and then allowed to cool to room temperature;
4) The API (metronidazole) was added and mixed for 1 hour using a Silverson mixer/homogenizer for uniform suspension;
5) Vials were filled with the suspension; and
6) Terminally sterilized by gamma irradiation.

The compositions described in Table 1 were prepared.

TABLE 1

| | | | | | Metronida- |
|---|---|---|---|---|---|
| | | SAIB | EA | MCT | zole |
| | Vehicle | (% w/w) | (% w/w) | (% w/w) | (% w/w) |
| 1 | SAIB:EA (85:15) | 63.75 | 11.25 | 0 | 25 (nominal) |
| 2 | SAIB:EA (85:15) | 56.1 | 9.9 | 0 | 34 (nominal) |
| 3 | SAIB:EA (80:20) | 52.8 | 13.2 | 0 | 34 (nominal) |
| 4 | SAIB:EA (70:30) | 44.8 | 19.2 | 0 | 36 (nominal) |
| 5 | SAIB:EA (75:25) | 48 | 16 | 0 | 36 (nominal) |
| 6 | Sustane:EA | 42.24 | 13.2 | 10.56 | 34 (nominal) |
| | (64:16:20) | | | | |
| 7 | Sustane:EA | 36.96 | 19.8 | 9.24 | 34 (nominal) |
| | (56:14:30) | | | | |
| 8 | Sustane:812:EA | 46.2 | 13.2 | 6.6 | 34 (nominal) |
| | (70:10:20) | | | | |
| 9 | SAIB:EA (80:20) | 52.6 | 13.2 | 0 | 34 (nominal) |

SAIB = sucrose acetate isobutyrate; EA = ethyl acetate; MCT = medium chain triglycerides; Sustane = Sustane ™ SAIB MCT Food Grade (80:20 SAIB:MCT); 812 = MIGLYOL ® 812N.

### Example 2: Formulation Development

Formulation development studies included evaluation of the impact of particle size on physical properties, scalability and mixing for commercialization. A High Shear Silverson mixer was used to mix formulations containing 36% drug load in 80:20 SAIB:EA and 75:25 SAIB:EA using three different metronidazole API lots of varying particle sizes ranging from D₉₀ of 12 microns to 119 microns. The formulations prepared with micronized (D₉₀ 12 micron and 21 micron) API were too viscous and lacked the flow properties to withdraw from a single vial formulation. The formulations prepared with non-micronized API had good flow properties and were capable of being withdrawn from sealed vials. The API particle size was determined to be a critical parameter for the formulations as well as having a marked impact on the release rate (in-vitro). Smaller API particle size resulted in higher viscosity and slow releasing formulations (in-vitro), and larger API particle size resulted in less viscous and faster releasing formulas with high drug bursts (in-vitro). The use of the High Shear Silverson mixer tended to have some effect on the particle size distribution, resulting in a lower burst and a more continuous release profile. Two formulas prepared with the High Shear Silverson mixer were chosen for evaluation in a PK study in dogs and stability/IVR studies.

Two formulations, i.e. 36% DL (80/20 SAIB:EA & 36% DL (75/25 SAIB:EA), were prepared for a PK study in dogs. Both formulas were prepared as 100 mL batches using non-micronized Corden Material (D₉₀=119 micron) and the High Shear Head on the Silverson Mixer. Each formulation was filled as a multidose presentation by adding approximately 7 mL of the suspension to a 10 mL glass borosilicate vial. Each vial was stoppered with a 20 mm plug stopper and sealed with an aluminum flip cap seal. Vials, stoppers and seals were purchased from Afton Scientific as part of Ready to Fill Kits, ITem RTF8420: 10 mL, 20 mm Kit. The stoppers were Fluoro Tee coated to ensure minimal interference from any product contact.

The vials containing the complete mixed formula were subject gamma irradiation. Once irradiated, 5 vials of each formula were used for the PK study and 7 vials were retained for stability evaluation. The suspension settled during the delay in shipping the samples and gamma irradiation process so resuspension was required prior to dosing. No less than 30 seconds of vortexing was recommended. The PK laboratory noted that the vials required nearly three minutes of manual shaking and vortexing to resuspend the material.

In response to the difficulties suspending the formulas at the PK study site, studies were performed to evaluate resuspension of the material on the stability studies. Using vigorous shaking, irradiated formulation in the 80:20 SAIB:EA vehicle was slightly more difficult to resuspend than irradiated formulation in the 75:25 SAIB:EA vehicle. Non-irradiated samples are easy to resuspend manually.

### Stability Study

Six (6) vials from both formulations were placed on stability after Gamma irradiation. The vials were split between the 25° C./60% RH and 40° C./75% RH storage conditions. Appearance, Drug Load, Related Substances, and IVR were tested at 0, 1, 2 and 3 months. Appearance, Drug Load and Related Substances results appear to be not impacted by the storage conditions. The IVR results appear to be sensitive to temperature over time.

In order to investigate the effect of the vehicle and addition of organic solvents (DMSO, Methanol, and Ethyl Acetate) on the recovery of metronidazole, a recovery check was performed. Metronidazole API, used as a reference standard, was dissolved in diluent (80:10 Water:Methanol) and added to the second dilution step prior to the crashing of the SAIB using the aqueous diluent. The sample was then diluted to volume using the same diluent. The vehicle crashed out of solution and the samples were filtered with a 0.45 micron PTFE filter and injected onto the system. The recovery of metronidazole in all three solvents was 100%+1%.

Next, samples were prepared which contained actual formulations at 36% Drug Load. Due to negative peaks in the ethyl acetate chromatograms, only DMSO and methanol samples were prepared. Both samples recovered the theoretical concentration of metronidazole used to formulate at 100±2%.

The major degradant found for metronidazole in compendial references was USP Related Compound A (2-methyl-5-nitroimidazole). A solution containing 1% of the running concentration (0.001 mg/mL) of the 2-methyl-5-nitroimidazole (“2M5N”) and 0.1 mg/ml metronidazole was prepared and injected onto the system. The 2M5N was resolved from the metronidazole with a resolution of 2.6. A series of dilutions was prepared to determine the recovery of 2M5N from the metronidazole standard, linear curve of 2M5N, and against a singular Response Factor of 2M5N. Recovery against the metronidazole standard was generally high. The 2M5N curve and 0.5% Response Factor recoveries were comparable. Metronidazole recovery was also not impacted by the presence of the 2M5N peak. Reproducibility at each level was acceptable.

In order to assess scalability of the formulations, a High Shear Silverson mixer was used to mix leading batches. Both Micronized and Non-Micronized metronidazole were initially evaluated in a 75:25 SAIB:EA vehicle at 36% Drug Load. The micronized material possessed poor flow characteristics with the high shear mixer. The non-micronized material displayed good flow characteristics but settled to the bottom of the container. Implants were prepared and assessed against the conventional Syringe Mixing technique.

IVR results for formulations prepared with the Silverson High Shear mixer demonstrated more continuous sustained release than formulations intermittently mixed with syringes. Additionally, the formulations prepared with non-micronized API were less viscous and easily withdrawn with a syringe than previous formulations using micronized APL. The physical properties of formulations using non-micronized API therefore could be amenable to being vialed for a potential multi-dose presentation.

Additional API was ordered with various D₉₀ particle size values to assess the best distribution of particles for product performance and presentation. Three materials were received (D₉₀=12.2, 21.2, and 119 micron) and formulated at 36% Drug Load in both 80:20 SAIB:EA and 75:20 SAIB:EA vehicles.

These six formulations were then tested for IVR. The release rate decreases and the initial burst gets lower as the D₉₀ particle size decreases; however, as the D₉₀ particle size decreases the viscosity of the formulation increases. The 80:20 SAIB:EA vehicle resulted in a lower burst, but similar slope of release. Based on the flowability and release characteristics of the 119 micron non-micronized material in both the 80:20 and 75:25 SAIB:EA vehicles, these formulations were chosen for further stability studies and PK study.

### Example 3: Gamma Irradiation Study

The formulations were compounded at 30% w/w drug load and 36% w/w drug load in a 75:25 vehicle (see Table 2). Half of the vials were overlaid with nitrogen in an attempt to alleviate discoloration during the irradiation process. Cooling using dry ice during irradiation was also examined. The nitrogen overlay seemed to have an intensifying effect on the discoloration and so only the air overlaid samples were used in PK studies in animals.

TABLE 2

| | | Metronida- | | Gamma | |
|---|---|---|---|---|---|
| | | zole | | Irradia- | Processing |
| | Vehicle | (% w/w) | Overlay | tion | Temp. |
| 10a | SAIB:EA (75:25) | 30 (nominal) | N₂ | 10 kGy | Cooled |
| 10b | SAIB:EA (75:25) | 30 (nominal) | Air | 10 kGy | Cooled |
| 10c | SAIB:EA (75:25) | 30 (nominal) | N₂ | 10 kGy | Non-Cooled |
| 10d | SAIB:EA (75:25) | 30 (nominal) | Air | 10 kGy | Non-Cooled |
| 11a | SAIB:EA (75:25) | 36 (nominal) | N₂ | 10 kGy | Cooled |
| 11b | SAIB:EA (75:25) | 36 (nominal) | Air | 10 kGy | Cooled |
| 11c | SAIB:EA (75:25) | 36 (nominal) | N₂ | 10 kGy | Non-Cooled |
| 11d | SAIB:EA (75:25) | 36 (nominal) | Air | 10 kGy | Non-Cooled |

### Gamma Level and Temperature Cycling Study

Formulation development studies included evaluation of the impact of Gamma irradiation strength and processing temperature during gamma irradiation. A single 36% drug load was prepared in a vehicle with a 75:25 SAIB:EA ratio. This batch was vialed and split into 9 groups. Six of the groups were shipped to Sterigenics for irradiation at varying levels (5, 10, 20 kGy) both cooled and uncooled during irradiation. The 7th group was sent as a control. The eighth and ninth groups were placed in appropriate chambers for a ‘freeze-thaw’ and ‘heat-cool’ study respectively. Each of these groups were cycled 3 times at the indicated temperatures.

All of the above samples were tested for Appearance, Resuspendability and In Vitro Release (IVR). The irradiated samples demonstrated a color change that intensified with the high irradiation levels. There was no discoloration observed in the heat/cool or freeze thaw studies. All samples were resuspended, however the irradiated samples took significantly more time (>60 seconds versus the 10-30 seconds for non-irradiated samples) than the non-irradiated samples. In Vitro Release demonstrated a ‘burst’ of material during the release for the irradiated samples. The higher irradiation level correlated with an earlier burst in the IVR. While this may be an artifact of the IVR method, as it is not seen in animal models in vivo, it may be linked to some type of degradation that occurs during the irradiation process. Temperature cycling had no effect on the non-irradiated samples. From the ladder study 10 kGy was chosen as the best level to go forward with additional studies for PK and formulation development.

### 10 kGy Study

A second study was initiated to examine the effect of drug load, nitrogen overlay and cooling during the gamma irradiation process. Two batches were made using the Silverson High Shear mixer. Both batches were formulated in the 75:25 SAIB:EA vehicle. One batch was formulated to contain 30% drug load (w/w) and the other was formulated to contain 36% drug load (w/w). These batches were then placed into vials and sealed, half under atmospheric air and half under nitrogen. The samples were processed at 10 kGy. Half of the vials were cooled with dry ice during the sterilization process and the other half were processed at room temperature. This led to a total of 8 groups for testing. Appearance testing determined that the vials treated with the nitrogen headspace had an increase in discoloration. The vials processed under air were then used for the animal PK study.

The vials were also tested for Appearance, Resuspendability and IVR. The appearance for the cooled process samples were tinged green post-irradiation and the non-cooled samples were tinged yellow. The time it took to resuspend the drug in solution increased post-irradiation (>60-70 see). The IVR for the samples were not differentiated from previous studies, with sporadic ‘rupture’ of the implant observed in some samples.

### Preparation for Animal PK Study

The two formulations described in Table 2 were prepared at ˜350 g batches using non-micronized Corden material (D₉₀=119 micron) and the High Shear Head on the Silverson Mixer. Both batches were formulated using a 75:25 SAIB:EA vehicle. One batch was formulated at 30% Drug Load (w/w) while the second was formulated at 36% Drug Load (w/w).

Each formulation was filled as a multidose presentation by adding approximately 7.5 mL of the suspension to a 10 mL glass borosilicate Type 1 vial. Each vial was stoppered with a 20 mm plug stopper and sealed with an aluminum flip cap seal. Half of the vials were sealed under nitrogen. Vials, stoppers and seals were purchased from Afton Scientific as Ready to Fill kits (RTF8420: 10 mL, 20 mm Kit). The kit contains 20 mm Type I Schott vials, 20 mm S10-F451, 4432/50 stoppers and 20 mm FlipOff TruEdge Royal Blue Matte seals. The stoppers are a chlorobutyl elastomer with Fluorotec coating to ensure minimal interference from any product contact.

The vials containing the complete mixed formula were subject to gamma irradiation at 10 kGy. Half of the four batches above were gamma irradiated with dry ice during the process. The second half of the vials were processed at room temperature.

Once irradiated, the vials were assessed for appearance and resuspendability. It was determined that the nitrogen overlaid vials resulted in increased discoloration of the formulas.

The vials required nearly one to two minutes of manual shaking to resuspend the material. A total of six (6) dogs were administered the drug product at the target dose of 125 mg/kg for each of the 4 formulations (10b, 10d, 11b, and 11d) listed in Table 2.

The PK study resulted in a decreased burst, extended release and better repeatability for formulation 11b. The 36% drug load in 75:25 SAIB:EA with air overlay and dry ice gamma irradiation processing was chosen for additional studies.

### Conclusions

The optimal level of gamma irradiation for optimal In Vitro Release performance is 10 kGy with dry ice for cooling during processing. Discoloration post-gamma irradiation is affected by level of Gamma irradiation, nitrogen overlay and presence of dry ice during processing.

### Example 4: PK Study 1-Pharmacokinetics of Six Long-Acting Metronidazole Formulations Given Subcutaneously in Dogs

### Summary

The objective of this randomized, parallel, non-GLP pilot pharmacokinetic study was to evaluate safety and pharmacokinetics (PK) data for six long-acting formulations of metronidazole following a single subcutaneous administration to Beagle dogs.

18 healthy female Beagle dogs were used during this study. Each group of 3 dogs was dosed subcutaneously in the interscapular region with one of six metronidazole formulations:
Formula 1 (Group 1): —36% (nominal); 37.5 (actual) (w/w) Metronidazole in 70:30 SAIB:EA;
Formula 2 (Group 2): —36% (nominal); 37.2 (actual) (w/w) Metronidazole in 75:25 SAIB:EA;
Formula 3 (Group 3): —34% (nominal) (w/w) Metronidazole in SAIB:MCT_{Sustane}:EA 64:16:20;
Formula 4 (Group 4): —34% (nominal) (w/w) Metronidazole in SAIB:MCTSustane:EA 56:14:30;
Formula 5 (Group 5): —34% (nominal) (w/w) Metronidazole in SAIB:MCT₈₁₂:EA 70:10:20; and
Formula 6 (Group 6): —34% (nominal) (w/w) Metronidazole in SAIB:EA 80:20.

Physical examinations were conducted during acclimation to ensure that all dogs were in good health prior to dosing. During the study twice daily health observations were conducted. Blood samples were collected at 30 min., 2, 4, 8, 12, 24, 48, 72, 96, 120, 144, and 168 hours after dose administration for determination of metronidazole concentration. Development of a hard knot at the injection site was expected due to the creation of an in-situ depot at the injection site. The injection sites were evaluated at pre-treatment, 30 minutes, 1, 2, 4, 12 hours (+15 min), 24, 48, 72, 96, 120, 144 and 168 hours (+2 hours) post dosing. At each time point, depot formation was recorded in the Electronic Data Capture system (EDC). Due to limitations of the EDC system, depot formation was recorded as edema. Heat and erythema were assessed using a modified Draize scoring system. Heat and pain at the injection site were evaluated. Further, the Length (L), Width (W) and Height (H) of the injection site depot were measured.

There were no findings on the pre-dose injection site observations. There were no treatment-related findings during clinical observations with the exception of findings at the dose site. Swelling and/or hardness (recorded as edema) at the injection site was expected due to the formation of an in-situ depot of the test article and these findings were present in all treatment groups. Upon palpation, there were no findings of pain or heat (warm to the touch) at any of the dose sites except for Animal 6501 (Formula 6) when heat was present in the PM on Dosing Phase Day 2. This finding was resolved at the next scheduled observation on the following morning and was not considered adverse due to the transient nature of the finding.

Although there was high individual animal variability in regard to the size of the palpable depot present for each dog, Group 3 (Formula 3) and Group 4 (Formula 4) animals appeared to have a larger palpable depot when compared to Groups 1, 2, 5, and 6 (Formulas 1, 2, 5, and 6, respectively). Slight swelling was evident at the injection site beginning at 0.5 to 2 hours post injection. This likely represented the initial injection volume. The swelling decreased and was not evident between 12-24 hours for most animals in all groups. The palpable depot began to increase in size between 24-36 hours in most animals. The depot remained palpable in some animals in all groups at the end of the study.

All six formulations were easily mixed and exhibited good syringeability. None of the formulations tested exhibited sustained release to 120 hours, and peak concentrations for all groups except Group 1 were higher than desired.

### Objective

The objective of this randomized, parallel, non-GLP pilot pharmacokinetic study was to evaluate safety and pharmacokinetics (PK) data for six long-acting formulations of metronidazole following a single subcutaneous administration to Beagle dogs.

### Study Design

Three healthy dogs per treatment group were dosed subcutaneously in the interscapular region with 125 mg/kg metronidazole. Each group was treated with one of six long-acting metronidazole formulations. Injection site and clinical observations were conducted on all animals at predose, 30 minutes, and 1, 2, 4, 24, 48, 72, 96, 120, 144, and 168 hours post-dose administration. Blood was collected at the same timepoints, processed to plasma, and sent to Pxyant Labs, Inc. (Colorado Springs, CO) for pharmacokinetic evaluation of metronidazole. Physical examinations were conducted during acclimation to determine that the dogs were in good health prior to the initial treatment. General health of the dogs was monitored twice daily with a minimum of 6 hours between observations beginning at acclimation.

### Materials and Methods

Dose formulation and injection volume by Group and individual animal are provided in Table 3.

TABLE 3

| | | Concentra- | Dose |
|---|---|---|---|
| | Group | tion | Level |
| Formulation | # | (mg/mL) | (mg/kg) |
| 36% (nominal)(w/w) Metronidazole in | 1 | 425 | 125 |
| 70:30 SAIB:EA | | | |
| 36% (nominal)(w/w) Metronidazole in | 2 | 425 | 125 |
| 75:25 SAIB:EA | | | |
| 34% (nominal)(w/w) Metronidazole in | 3 | 401 | 125 |
| 64:16:20 SAIB:MCT_{Sust.}:EA | | | |
| 34% (nominal)(w/w) Metronidazole in | 4 | 401 | 125 |
| 56:14:30 SAIB:MCT_{Sust.}:EA | | | |
| 34% (nominal)(w/w) Metronidazole in | 5 | 401 | 125 |
| 70:10:20 SAIB:MCT₈₁₂:EA | | | |
| 34% (nominal)(w/w) Metronidazole in | 6 | 401 | 125 |
| 80:20 SAIB:EA | | | |

Some settling and separation of the test article was observed in all formulations. The test article was mixed attaching a sterile 5 mL syringe to the test article syringe using a coupler, and mixing 5-10 cycles until the formulation was homogeneous. The dose was administered subcutaneously under the loose skin in the interscapular region with an 18-gauge needle. Syringes were weighed before and after dosing.

Injection site observations were conducted at 0 min (pre-dose), 30 min, 1, 2, 4, 24, 48, 72, 96, 120, 144, and 168 hours post dose-administration. Heat at the injection site was evaluated as present or absent (yes/no). Pain at the injection site was scored with the following criteria: 0=Absent, 1=Slight, 2=Significant. Swelling and/or hardness at the injection site was expected due to the formation of an in-situ depot of the test article. When the swelling and/or hardness was present, calipers were used to measure the two longest perpendicular axes in the x/y (width/length) plane, as well as the z axis (height) to the nearest 0.1 mm. A “palpable mass tracking” measurement was used to capture the dose site swelling measurements. The term, “palpable mass tracking”, was the program name within the electronic data capture system and refers to the palpable depot that was formed at the injection site post dose administration. Injection site reactions were evaluated using a modified Draize Scoring System as detailed in Table 4. Using this system, edema was not distinguished from the palpable depot.

TABLE 4
Individual Animal Draize Scores

| Category | Score | Description |
|---|---|---|
| Erythema | 0 | None |
| | 1 | Slight |
| | 2 | Well-defined |
| | 3 | Moderate or severe |
| | 4 | Severe or slight eschar formation |
| | | (injuries in depth) |
| Edema ^{A} | 0 | None |
| | 1 | Very slight |
| | 2 | Slight (well-defined edges) |
| | 3 | Moderate (raised >1 mm) |
| | 4 | Severe (raised >1 mm and extending |
| | | beyond the area of exposure) |

^{A} Edema could not be distinguished from the palpable depot formed at the injection site post dose administration

Blood samples were collected by venipuncture from the jugular vein of each dog at 0.5, 2, 4, 8, 12, 24, 24, 72, 96, 120, 144, and 168 hours following dosing. Scheduled and actual blood collection times were recorded onto prepared forms. Actual blood collection times could vary by +10% of the scheduled time point to accommodate sample collection.

Samples were collected into labeled tubes containing K2EDTA (3 mL) and centrifuged for the collection of plasma. Samples were placed on wet ice/ice packs before being centrifuged at 3,000 rpm for 15 minutes at 4° C. within 1 hour of sample collection. The plasma samples were then aliquoted into a prelabeled primary and backup cryovial and temporarily stored on dry ice until frozen storage (−50 to −90° C.). Samples were shipped on dry ice to Pyxant Laboratories for analysis.

### Bioanalysis

Metronidazole was analyzed in plasma samples at Pyxant Laboratories, Inc. by an acidified methanol protein crash and quantitated against a freshly prepared calibration curve using a best fit regression from 1.0 to 2,000 ng/mL. During the first assay, 154/216 samples were above the limit of quantiation. Therefore, the samples were diluted and reanalyzed.

### Results and Discussion

In-life phase: All dogs were healthy during the study. No serious adverse events or animal deaths.

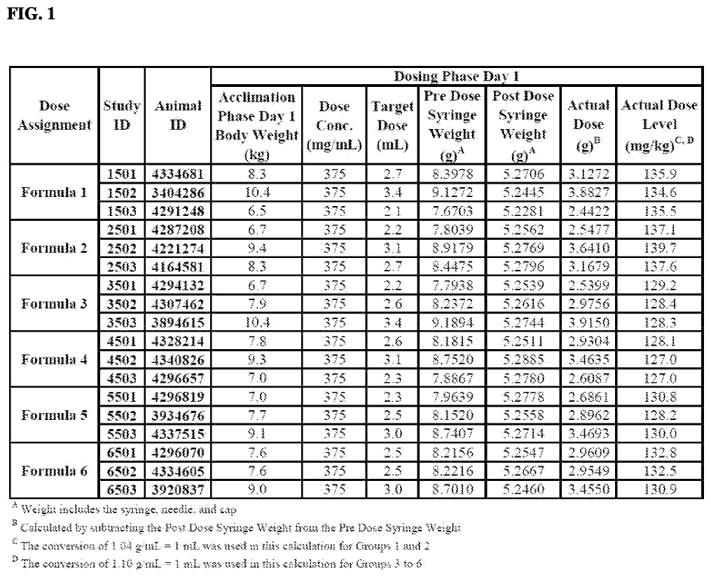

Syringe weights and dose: All individual animal predose and postdose syringe weights are in FIG. 1. Actual dose amount was calculated by subtracting the post dose syringe weight from the pre-dose syringe weight using a using a conversion of 1.04 g/mL=1 mL for Groups 1 and 2 and a conversion of 1.10 g/mL=1 mL for Groups 3 through 6. In all instances, the calculated dose amounts were higher than the target dose which resulted in a dose level that was higher than the target by 12 to 17% (140.0 to 146.1 mg/kg).

Clinical Observations: There were no treatment-related findings with the exception of findings at the dose site described in the next section.

Scabs at the dose site were observed for Animals 2502 (Formula 2), 6501 (Formula 6), and 6502 (Formula 6). This is a common finding at injection sites and is not considered to be test article related. Scabs, erythema, edema, and absent hair at locations unrelated to the dose site, thin appearance, and discharge from the eyes were observed during acclimation as well as the dosing phase. These findings are common observations in dogs and are not considered to be treatment-related.

Injection site observations: Swelling and/or hardness (recorded as edema) at the injection site was expected due to the formation of an in-situ depot of the test article and these findings were present in all treatment groups. Upon palpation, there were no findings of pain or heat at any of the dose sites with the exception of Animal 6501 (Formula 6) when heat was present in the PM on Dosing Phase Day 2. This finding was resolved at the next scheduled observation on the following morning and was not considered adverse due to the transient nature of the finding.

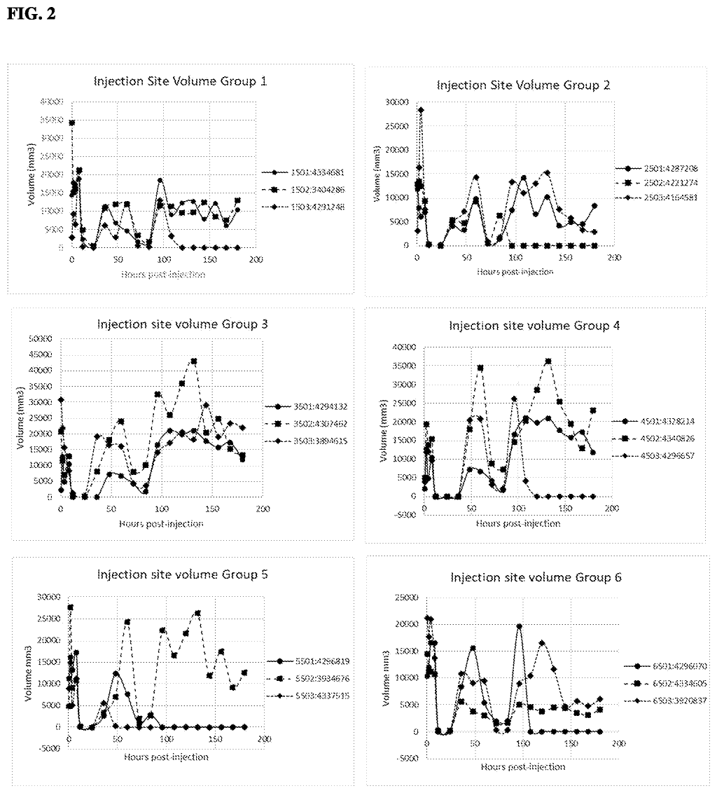

A palpable depot at the dose site was present in all dogs regardless of treatment group following administration of test article. All treatment groups had 2/3 dogs with a palpable depot present on Dosing Phase Day 7 except for Group 5 (Formula 5) which had 1/3 dogs with a palpable depot present and Group 3 (Formula 3) which had 3/3 dogs with a palpable depot present. Although there was high individual animal variability in regard to the size of the palpable depot, Group 3 (Formula 3) and Group 4 (Formula 4) appeared to have larger palpable depots when compared to Groups 1, 2, 5, and 6 (Formulas 1, 2, 5, and 6, respectively). Injection site volumes by group are provided in FIG. 2.

There were no findings of erythema in Groups 3, 4, and 5 (Formulas 3, 4, and 5, respectively). Maximal Draize scores for erythema were a 2 for Groups 1 and 6 (Formulas 1 and 6, respectively) and 1 for Group 2. Maximal Draize scores for edema were 3 for Groups 2, 4, 5, and 6 (Formulas 2, 4, 5, and 6, respectively) and 4 for Groups 1 and 3 (Formulas 1 and 3, respectively). Edema, which could not be distinguished from the palpable depot formed at the injection site, was noted on Dosing Phase Day 1 following administration of test article and continued for most animals correlating with palpable depot measurements.

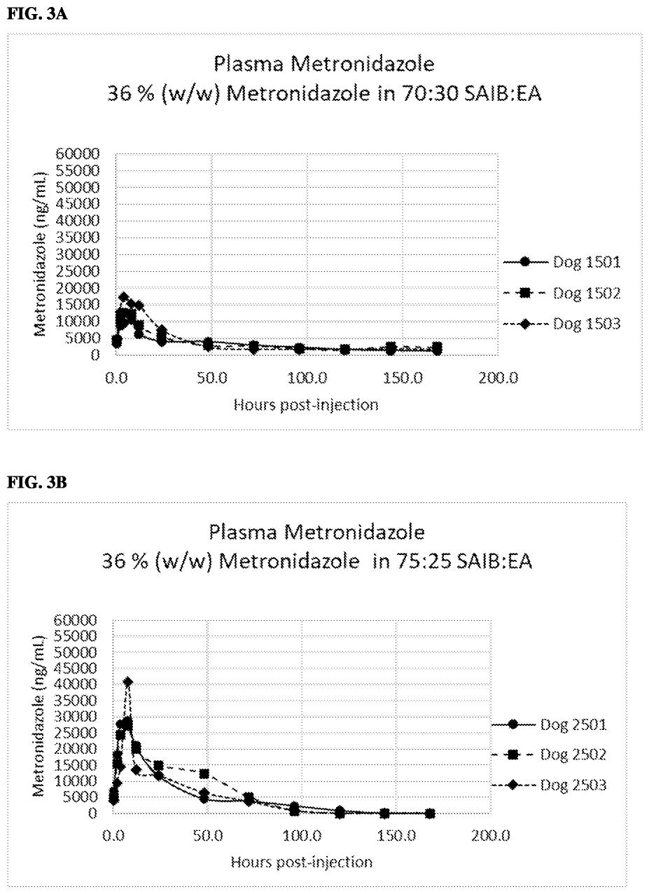

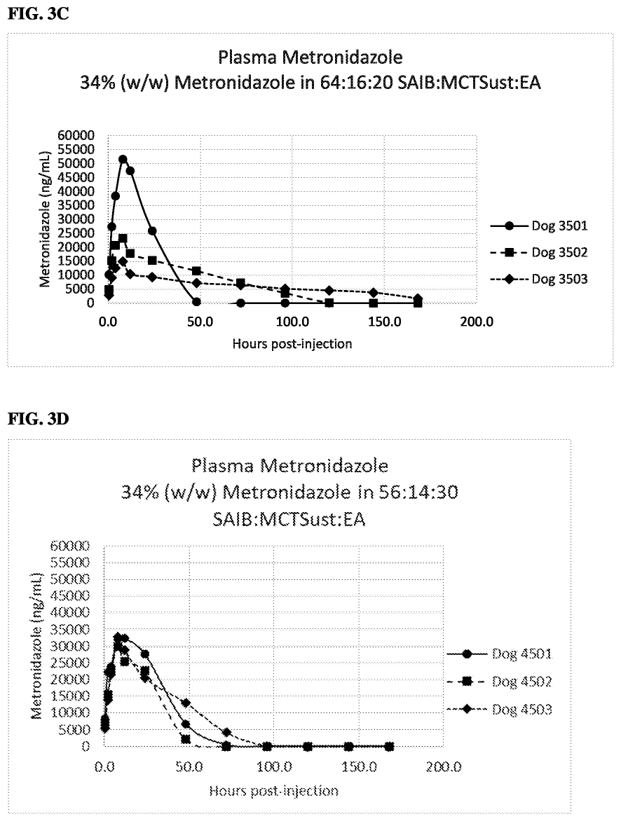

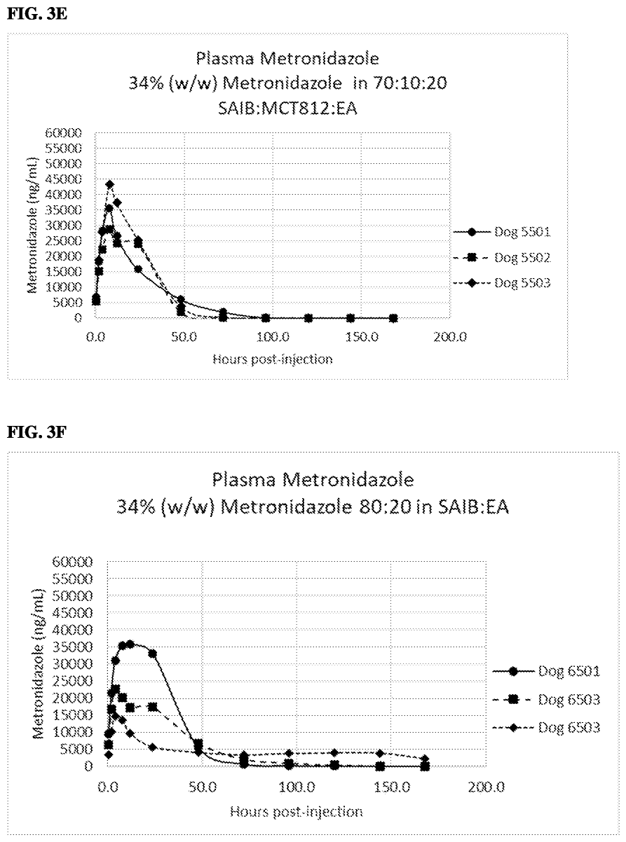

Individual dog plasma metronidazole concentration by timepoint and group are presented in FIGS. 3A-3F.

### Conclusions

Based on the size of the palpable depot, injection site findings, and release characteristics, Formulas 2 and 6 appear to be most promising and were developed and tested further.

### Example 5: PK Study 2-Pharmacokinetics and Safety of Two Long-Acting Formulations of Metronidazole in Dogs

### Summary

The objective of this non-GLP pilot study was to evaluate safety and obtain bioanalytical data to assess the pharmacokinetics of two long-acting formulations of metronidazole following a single subcutaneous administration to beagle dogs.

The in-life portion of this study was conducted according to an unmasked, single period, 2-group, parallel design. Five male Beagle dogs were blocked by body weight and randomly assigned to receive either Formula 1 (80:20 SAIB:EA, 36% (nominal), 37.5% (actual) (w/w) metronidazole) or Formula 2 (75:25 SAIB:EA, 36% (nominal), 37.2% (actual) (w/w) metronidazole) in groups 1 and 2 respectively.

On Day 0, dogs were injected subcutaneously in the intrascapular space with of one of the two formulations of metronidazole. The dose was 62.5 mg/kg of metronidazole (0.14 mL/kg of suspension). Both suspensions were difficult to resuspend prior to dosing, requiring over three minutes of vortexing and shaking to achieve a homogenous suspension.

Physical examinations were conducted on all dogs once on the first day of acclimation and body weights were measured on days-1 and day 10. General health observations were performed daily starting on the first day of acclimation and continuing through the end of the study. Dogs were observed twice daily for the first 48 hours after dosing.

Application sites were observed for local tolerance starting after treatment on day 0 at time points 0.5, 1, 2, 6, 12, 24, 48, 72, 96, 120, 144, 168, 192, 216, and 240 hours post-injection. Injection sites were evaluated for erythema, pain on palpation, edema, and the presence of heat at the injection site. Blood samples for PK analysis were collected from all dogs pre-dose and at 0.25, 0.5, 0.75, 1, 1.5, 2, 2.5, 3, 4, 6, 8, 12, 24, 48, 72, 96, 120, 144, 168, 192, 216, and 240 hours following day 0 dose administration. Blood samples were processed to plasma and stored frozen at −70° C. or colder until shipped to Pyxant for bioanalysis.

There were no abnormal clinical observations during the study. Four out of five dogs in each group displayed minor injection site reactions. The most common was edema (3 dogs in each group), followed by heat at the injection site (two in Group 1 and 1 in Group 2) and erythema (2 in Group 2).

The overall curve shapes between the two formulas were very similar, a large initial burst lasting until approximately 12 hours after dosing, and with the majority of the release occurring within the first 48 hours. Although Formula 1 showed less variability in Cₘₐₓ than Formula 2), Formula 2 demonstrated a longer, shallower release curve past 48 hours.

The two injectable extended-release formulations of metronidazole tested demonstrated similar release profiles, with less variability seen in Formula 1. However, considering the longer, shallower release curve demonstrated by most dogs receiving Formula 2, it was decided that Formula 2 would be chosen for further development.

### Objective

The objective of this non-GLP pilot study was to evaluate safety and obtain bioanalytical data to assess the pharmacokinetics of two long-acting formulations of metronidazole following a single subcutaneous administration to beagle dogs.

### Results and Discussion

Dosing—The metronidazole injectable extended-release suspension was administered via subcutaneous injection into the intrascapular region. The dose groups and formulations administered on day 0 are listed in Table 5:

TABLE 5

| | Number of | | |
|---|---|---|---|
| Group | Animals | Treatment | Formulation |
| 1 | 5 | Formula 1 | 64% 80:20 SAIB:EA, 36% (nominal), |
| | | | 37.5% (actual) (w/w) Metronidazole |
| 2 | 5 | Formula 2 | 64% 75:25 SAIB:EA, 36% (nominal), |
| | | | 37.2% (actual) (w/w) Metronidazole |

Both formulas required over three minutes of vortexing and shaking to achieve a homogenous suspension and samples were returned to the manufacturer for follow-up. Doses were dispensed by volume, but dosing syringes were weighed before and after treatment to determine the actual dose administered to each animal. The actual administered dose extrapolated to mg/kg body weight (bw) ranged from 54.9 mg/kg to 67.8 mg/kg. A complete list of administered doses on a mg/kg bw basis can be found in Table 6.

TABLE 6

| | | | Syringe | Syringe | Wt. Test | Total API | |
|---|---|---|---|---|---|---|---|
| | | B.W. | Wt. | Wt. | Article | (mg), based | mg/kg |
| | Dog | Day −1 | Before | After | Admin | on nominal | bw |
| Treatment | ID | (kg) | (g) | (g) | (g) | concentration | dosed |
| 1 | 166003 | 12.085 | 6.563 | 4.620 | 1.943 | 699.5 | 57.9 |
| | 166120 | 11.770 | 6.654 | 4.610 | 2.044 | 735.8 | 62.5 |
| | 165819 | 10.100 | 6.357 | 4.601 | 1.756 | 632.2 | 62.6 |
| | 166116 | 10.860 | 6.500 | 4.601 | 1.899 | 683.6 | 63.0 |
| | 166016 | 11.115 | 6.592 | 4.641 | 1.951 | 702.4 | 63.2 |
| 2 | 166104 | 11.855 | 6.444 | 4.637 | 1.807 | 650.5 | 54.9 |
| | 166008 | 10.055 | 6.317 | 4.627 | 1.690 | 608.4 | 60.5 |
| | 166108 | 11.005 | 6.655 | 4.668 | 1.987 | 715.3 | 65.0 |
| | 165813 | 10.505 | 6.562 | 4.619 | 1.943 | 699.5 | 66.6 |
| | 166102 | 10.685 | 6.694 | 4.682 | 2.012 | 724.3 | 67.8 |

Safety-No abnormal general health observations were made for any dog during the study. Four total dogs receiving Formula 1 displayed injection site reactions; heat and edema was noted at the injection site for one dog, one additional displayed heat only, and two additional dogs displayed edema only. Four total dogs receiving Formula 2 also displayed injection site reactions; one dog displayed heat, erythema, and edema at the site, one additional showed erythema only, and two additional displayed edema only. These were considered mild injection site reactions and were well tolerated.

All animals were of a healthy body weight at the time of dosing. Because of the length of the study, no post-study weights were measured, and no potential changes in body weight were likely that would interfere with the interpretation of results.

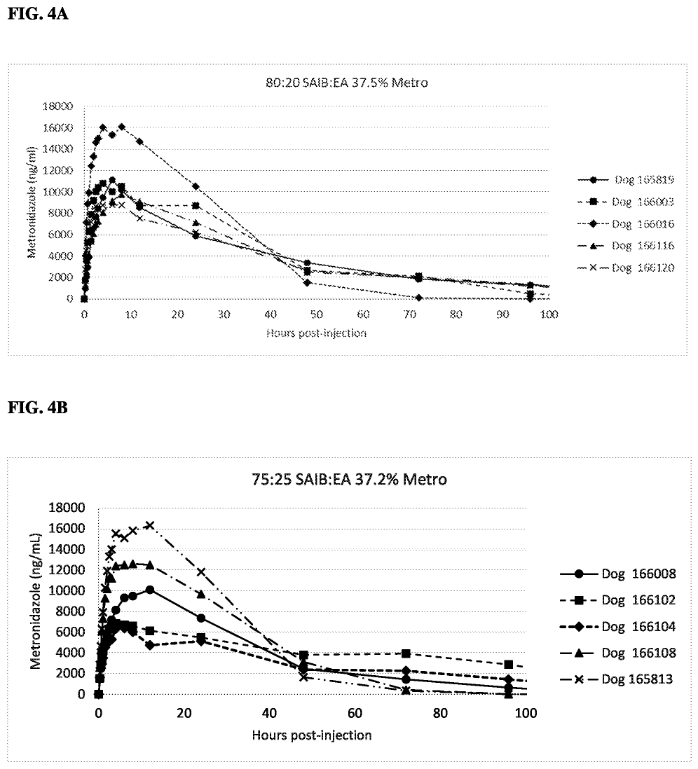

Bioanalysis-Graphs of individual animal concentration profiles in Group 1 and Group 2 are shown in FIGS. 4A and 4B, respectively. The lower limit of quantitation (LLOQ) was 1.00 ng/mL.

Pharmacokinetics—The Tₘₐₓ and Cₘₐₓ for the concentration x time curves for individual dogs can be seen in Table 7.

TABLE 7

| Formula | Dog ID | Tₘₐₓ (hr) | Cₘₐₓ (ng/mL) |
|---|---|---|---|
| 1 (80:20 SAIB/EA) | 165819 | 6 | 11100 |
| | 166003 | 4 | 10800 |
| | 166016 | 8 | 16100 |
| | 166116 | 8 | 9770 |
| | 166120 | 4 | 8780 |
| 2 (75:25 SAIB/EA) | 166008 | 12 | 10100 |
| | 166102 | 4 | 6830 |
| | 166104 | 6 | 6370 |
| | 166108 | 8 | 12600 |
| | 165813 | 12 | 16300 |

Individual curves from both formulations were very similar in overall shape with an initial burst, reaching a Tₘₐₓ between 4 and 12 hours after dosing. Formula 2 showed more variability in Tₘₐₓ, ranging from 4 to 12 hours vs. 4 to 8 hours for Formula 1. The range in Cₘₐₓ in Formula 1 is larger than seen in Formula 2 (6370 to 16300 ng/mL vs. 8780 to 16100 ng/ml respectively). The Cₘₐₓ values for Formula 1 were tightly clustered (8780 to 11100 ng/ml) with exception of the Cₘₐₓ for Dog 166016 (16100 ng/ml).

When only the first 96 hours of the time concentration curve are considered most curves in both groups showed a relative plateau in concentration between 4 and 12 hours, with an almost linear between 12 and 48 hours, flattening into a long shallow linear release.

Despite the higher variability in Cₘₐₓ displayed by Formula 2, on the average, it demonstrated a longer, shallower sustained release past 48 hours in more dogs. Given the overall similarity in in vivo release profile, it was decided to pursue Formula 2 for further development.

### Conclusions

The two injectable extended-release formulations of metronidazole tested demonstrated similar release profiles, with less variability seen in Formula 1. However, considering the longer, shallower release curve demonstrated by most dogs receiving Formula 2, it was decided that Formula 2 would be chosen for further development.

### Example 6: PK Study 3-Pharmacokinetics and Safety of Two Long-Acting Formulations of Metronidazole in Dogs

### Summary

The in-life portion of this study was conducted according to a single period, 4-group, parallel design. This study was unmasked. This should not have introduced any bias as it was an exploratory study with no control group.

Twenty-four Beagle dogs (12 male and 12 female) were blocked by body weight and randomly assigned to one of four dose groups.

Two dose groups were administered a formulation containing 70%75:25 SAIB:EA, 30% Metronidazole, either cooled during irradiation or not cooled during gamma irradiation at 10 kGy. The remaining two groups were administered formulations containing 64%75:25 SAIB:EA, 36% Metronidazole, either cooled or not cooled during gamma irradiation at 10 kGy.

On Day 0, dogs were injected subcutaneously in the intrascapular space with the assigned formulations of metronidazole. The dose was 125 mg/kg of metronidazole (0.38 mL/kg of suspension). Vials were mixed by vortexing for approximately 30 seconds to resuspend prior to administration and were inverted by hand immediately prior to drawing the dose. The syringe plunger froze up while injecting one dog in group B, and the dog did not receive approximately 0.3 mL of the calculated intended dose, representing and under-dosage of less than 10%.

Physical examinations were conducted on all dogs once during of acclimation and body weights were measured at the beginning of acclimation and again on day-1 and day 10. General health observations were performed daily starting on the first day of acclimation and continuing through the end of the study. Dogs were observed twice daily for the first 48 hours after dosing.

Injection sites were observed for local tolerance starting after treatment on day 0 at time points 0.5, 1, 2, 6, 12, 24, 48, 72, 96, 120, 144, 168, 192, 216, and 240 hours post-injection. Injection sites were evaluated for erythema, pain on palpation, edema, and the presence of heat at the injection site.

Blood samples for PK analysis were collected from all dogs pre-dose and at 0.5, 1, 2, 3, 4, 6, 8, 12, 24, 48, 72, 96, 120, 144, 168, 192, 216, and 240 hours following day 0 dose administration. Blood samples were processed to plasma and stored frozen at −70° C. or colder until shipped to Pyxant for bioanalysis.

There were no abnormal clinical observations during routine general health observations made during the study. One dog in group D experienced a nontreatment related adverse event that required removal from the study on day 7.

Minor injection site reactions were seen in all dogs in all groups, excepting one dog in Group A. These reactions were self-limiting and overall the injections were well tolerated.

The concentration curves for all four groups demonstrated that all the formulations tested showed increased release duration and a lower initial burst that seen previously.

The curves were very similar between the 30% and 36% drug loads, indicating that drugload is not a factor in duration or burst.

Plasma levels over 1000 ng/ml were seen out to 120 hrs in all groups, with group C having the most dogs (4/6) at or above that level at that time. In a previous study, Example 4, only 1/3 dogs had levels over 1000 ng/mL at 96 hours when given the same dose. The site of action for this drug is the intestinal fluids and metronidazole is highly distributed from the plasma compartment to the intestinal fluids after subcutaneous injection, thus plasma levels can be used to gauge duration in the intestinal fluids.

Cₘₐₓ varied from 16,900 to 26,100 ng/mL as compared to a prior Cₘₐₓ range of 27,200 to 40,800 ng/mL when given the same dose of an earlier, similar formulation (see Example 4).

Given the similarity in curve shape between the 30% and 36% drug loads, the 36% drugload was chosen for further development. Considering that cooling during gamma irradiation is a standard practice and given that the duration of drug exposure in the plasma was greater in Group C, this condition was chosen for further development.

### Objective

The objective of this non-GLP pilot study was to evaluate safety and obtain bioanalytical data to assess the pharmacokinetics of four long-acting formulations of metronidazole following a single subcutaneous administration to beagle dogs. The formulations compared a 30% drug load to the previous 36% drug load formulations and tested a cooled and non-cooled gamma irradiation sterilization process at 10 kGy to compare to the 25 kGy gamma irradiation process used in previous formulations (Examples 4-5).

### Results and Discussion

Dosing—The metronidazole injectable extended-release suspension was administered via subcutaneous injection into the intrascapular region. The dose groups are listed in

TABLE 8

| | Number | | Drug Load/ | | |
|---|---|---|---|---|---|
| | of | Treatment | irradiation | | Dose |
| Group | Animals | Day 0 | condition | Dose | Volume |
| A | 6 | Formula 2 | 30% (nominal) | 125 | 0.38 |
| | | | 33.2 (actual)/ | mg/kg | mL/kg |
| | | | cooled | | |
| B | 6 | Formula 4 | 30% (nominal) | 125 | 0.38 |
| | | | 32.5 (actual)/ | mg/kg | mL/kg |
| | | | non-cooled | | |
| C | 6 | Formula 6 | 36% (nominal) | 125 | 0.29 |
| | | | 38.9 (actual)/ | mg/kg | mL/kg |
| | | | cooled | | |
| D | 6 | Formula 8 | 36% (nominal) | 125 | 0.29 |
| | | | 39.2 (actual)/ | mg/kg | mL/kg |
| | | | non-cooled | | |

Dose group assignments and details regarding dose administration can be found in Table 9. All formulations were resuspended by approximately 30 seconds of vortexing. Doses were dispensed by volume based on the nominal concentration of the test article, but dosing syringes were weighed before and after treatment to determine the actual dose administered to each animal. The dose in grams administered to each dog can be found in Table 9. The actual administered dose of metronidazole based on the assay results and extrapolated to mg/kg body weight (bw) ranged from 119.71 mg/kg to 143.81 mg/kg.

TABLE 9

| | | | | Pre- | | | Post- | | |
|---|---|---|---|---|---|---|---|---|---|
| | | | | dose | | | dose | Treatment | |
| Treatment | | Animal | Dose | Syringe | | | Syringe | Completed | |
| Order/ | Treatment | Weight | Volume | Weight | Dose | Treatment | Weight | per | |
| Animal ID | Group | (kg) | (mL) | (g) | Route | Time | (g) | Protocol. | Comments |
| 1/245998 | A | 8.955 | 3.2 | 8.894 | SQ^{a} | 07:12 | 5.015 | Yes | Vial 1 |
| 2/307676 | A | 8.380 | 3.0 | 8.556 | SQ | 07:14 | 5.004 | Yes | Vial 2 |
| 3/244448 | A | 9.015 | 3.2 | 8.805 | SQ | 07:16 | 4.973 | Yes | Vial 3 |
| 4/307673 | A | 9.410 | 3.4 | 8.779 | SQ | 07:19 | 4.999 | Yes | Vial 4 |
| 5/166008 | A | 10.675 | 3.8 | 9.399 | SQ | 07:21 | 4.993 | Yes | Vial 5 |
| 6/689040 | A | 10.565 | 3.8 | 9.335 | SQ | 07:23 | 4.995 | Yes | Vial 6 |
| 7/872145 | B | 7.860 | 2.8 | 8.228 | SQ | 07:25 | 4.998 | Yes | Vial 1 |
| 8/962879 | B | 9.505 | 3.4^{b} | 8.942 | SQ | 07:27 | 5.441 | Yes | Vial 2^{b} |
| 9/962865 | B | 9.265 | 3.3 | 8.888 | SQ | 07:29 | 4.985 | Yes | Vial 3 |
| 10/689118 | B | 9.010 | 3.2 | 8.753 | SQ | 07:31 | 4.991 | Yes | Vial 4 |
| 11/689452 | B | 9.670 | 3.5 | 9.047 | SQ | 07:33 | 4.984 | Yes | Vial 5 |
| 12/307677 | B | 10.710 | 3.8 | 9.353 | SQ | 07:35 | 5.013 | Yes | Vial 6 |
| 13/872141 | C | 8.690 | 2.6 | 8.128 | SQ | 07:37 | 5.001 | Yes | Vial 1 |
| 14/962877 | C | 8.075 | 2.4 | 7.869 | SQ | 07:39 | 4.987 | Yes | Vial 2 |
| 15/235571 | C | 9.100 | 2.7 | 8.253 | SQ | 07:41 | 5.007 | Yes | Vial 3 |
| 16/307682 | C | 8.620 | 2.5 | 8.000 | SQ | 07:43 | 4.986 | Yes | Vial 4 |
| 17/728945 | C | 9.985 | 2.9 | 8.459 | SQ | 07:45 | 5.019 | Yes | Vial 5 |
| 18/962861 | C | 10.530 | 3.1 | 8.742 | SQ | 07:47 | 5.003 | Yes | Vial 6 |
| 19/314860 | D | 7.950 | 2.3 | 7.766 | SQ | 07:49 | 5.002 | Yes | Vial 1 |
| 20/570470 | D | 8.500 | 2.5 | 8.065 | SQ | 07:51 | 5.008 | Yes | Vial 2 |
| 21/307672 | D | 8.895 | 2.6 | 8.109 | SQ | 07:53 | 4.990 | Yes | Vial 3 |
| 22/043713 | D | 9.640 | 2.8 | 8.331 | SQ | 07:55 | 4.968 | Yes | Vial 5 |
| 23/033845 | D | 9.930 | 2.9 | 8.514 | SQ | 07:57 | 4.994 | Yes | Vial 6 |
| 24/166118 | D | 9.765 | 2.9 | 8.397 | SQ | 07:59 | 4.982 | Yes | Vial 8 |

^{a}SQ = subcutaneous
^{b}Not complete dose. Syringe froze, did not receive ~0.3 mL of dose.

Post-sterilization assayed concentration of experimental batches administered is found in Table 10.

TABLE 10

| | | Nominal | Assayed |
|---|---|---|---|
| | | Concentration | Concentration |
| Group | Batch No. | (mg/mL) | (mg/mL) |
| A | 22.05.VT2 | 350 | 388.3 |
| B | 22.05.VT4 | 350 | 364.1 |
| C | 22.05.VT6 | 425 | 443.9 |
| D | 22.05.VT8 | 425 | 466.7 |

Safety—No abnormal general health observations were made for any dog during as a result of the routine study observations. All dogs experienced hardness at the injection site at one point during the study. This was attributed to the formation of the subcutaneous drug depot and was not unexpected. Four out of five dogs in Group A, and all five dogs in each of the other treatment groups displayed minor injection site reactions. The most common was edema (four out of five dogs in Group A, and all the dogs in the other treatment groups), followed by slight to very slight erythema (One dog from each treatment group). Well-defined erythema was seen in one dog from Group A. Overall, the observed reactions were minor and the test article was well tolerated.

All animals were of a healthy body weight at the time of dosing. One dog (Dog 16 (307682) assigned to Group C exhibited a 10% weight loss over the course of acclimation but had no clinical signs of disease and appeared in good health at the start of the study.

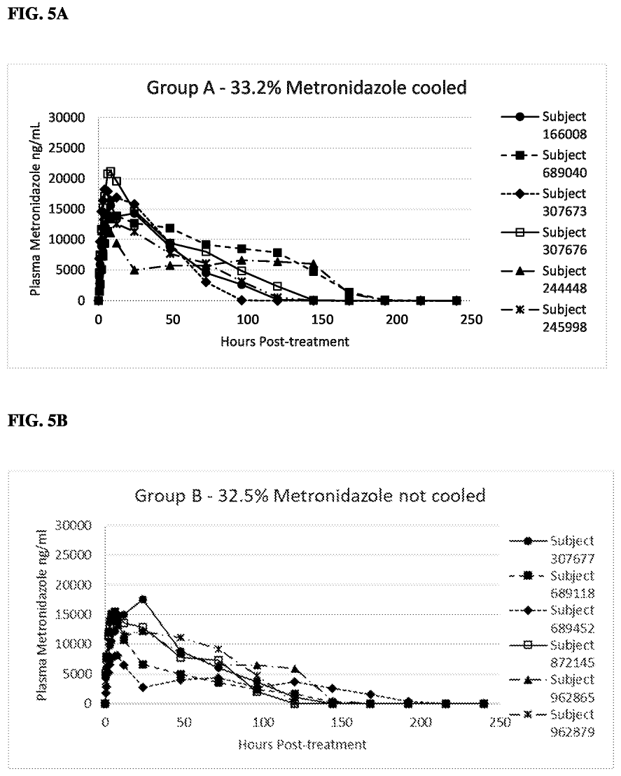

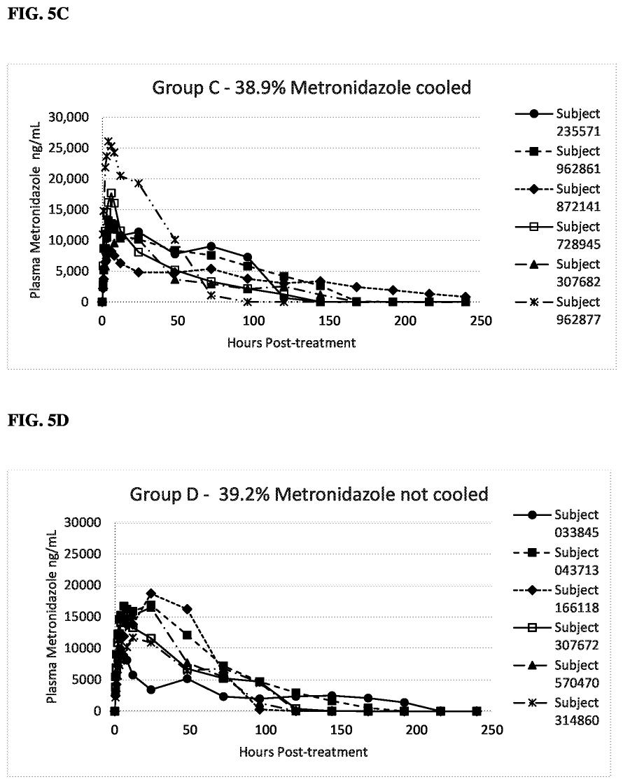

Bioanalysis-Graphs of individual animal concentration profiles in Groups A, B, C and D are shown in FIGS. 5A-5D, respectively.

Pharmacokinetics—The Tₘₐₓ and Cₘₐₓ for the concentration x time curves for individual dogs can be seen in Table 11.

TABLE 11

| Group | Dog ID | Tₘₐₓ (hr) | Cₘₐₓ (ng/mL) |
|---|---|---|---|
| A | 1/245998 | 6 | 14500 |
| A | 2/307676 | 8 | 21200 |
| A | 3/244448 | 6 | 11100 |
| A | 4/307673 | 4 | 18200 |
| A | 5/166008 | 8 | 15600 |
| A | 6/689040 | 12 | 13900 |
| B | 7/872145 | 6 | 15300 |
| B | 8/962879 | 4 | 14500 |
| B | 9/962865 | 4 | 14700 |
| B | 10/689118 | 6 | 15500 |
| B | 11/689452 | 8 | 8180 |
| B | 12/307677 | 24 | 17600 |
| C | 13/872141 | 6 | 8250 |
| C | 14/962877 | 4 | 26100 |
| C | 15/235571 | 6 | 12500 |
| C | 16/307682 | 12 | 10400 |
| C | 17/728945 | 6 | 17700 |
| C | 18/962861 | 4 | 13200 |
| D | 19/314860 | 12 | 11700 |
| D | 20/570470 | 24 | 16500 |
| D | 21/307672 | 8 | 14500 |
| D | 22/043713 | 24 | 16900 |
| D | 23/033845 | 4 | 9550 |
| D | 24/166118 | 24 | 18700 |

The concentration curves for all four groups demonstrated that all the formulations tested showed increased release duration and a lower initial burst than seen previously. Tₘₐₓ varied from 4 to 24 hours as compared to a maximum Tₘₐₓ of 12 hours seen in the earlier study (see Example 5). Cₘₐₓ varied from 16,900 to 26,100 ng/ml as compared to a prior Cₘₐₓ range of 27,200 to 40,800 ng/mL when given the same dose of an earlier, similar formulation (see Example 4). Most Cₘₐₓ values remained near or below 20,000 ng/ml with the exception of one dog in Group A (Dog 2/307676) with a Cₘₐₓ of 21,200 ng/ml and one dog in Group C (Dog 14/962877), who demonstrated a Cₘₐₓ of 26100 ng/ml There were no meaningful differences in overall curve shape between the 30% an 36% drug loaded formulations nor in those formulations that had been cooled during sterilization by irradiation and those that were not cooled.

Plasma levels over 1000 ng/mL were seen out to 120 hrs in all groups, with group C having the most dogs (4/6) at or above that level at that time. In a previous study, Example 4, only 1/3 dogs had levels over 1000 ng/mL at 96 hours when given the same dose. The site of action for this drug is the intestinal fluids and metronidazole is highly distributed from the plasma compartment to the intestinal fluids after subcutaneous injection, thus plasma levels can be used to gauge duration in the intestinal fluids.

### Conclusion

All four combinations of formulation and irradiation conditions tested demonstrated similar release profiles, with reduced initial burst and extended-release profiles as compared to previous studies. Given the similarity in curve shape between the 30% and 36% drug loads, the 36% drug load was chosen for further development. Considering that cooling during gamma irradiation is a standard practice and given that the duration of exposure was greater in Group C, this condition was chosen for further development despite this group having one dog with an elevated Cₘₐₓ compared to the other dogs in the study.

### Example 7: A Target Animal Pilot Dose-Finding and Preliminary Safety Study of an Extended-Release Formulation of Metronidazole at Five Dosages Given Subcutaneously Once or Twice for the Treatment of Giardia in Dogs

### Objective

The primary objective of the study is to determine the efficacious dose regimen(s) of Verté's injectable sustained release formulation of metronidazole for the treatment of susceptible infections of Giardia duodenalis in dogs as compared to a placebo preparation, using an induced infection model. Secondary objective includes evaluating the safety of the formulation when dosed within the range of dosages projected to be effective. The results will be used to select dosages for further evaluation in a dose determination study examining cysts and trophozoites and to determine an appropriate index of efficacy to power future studies. The present report focuses on the first objective.

### Study Design

This is a six-arm masked dose ranging study conducted in an induced disease model system. Infection with Giardia duodenalis was induced in 40 healthy dogs by inoculating each dog with 10,000 viable fecal cysts. Prior to infection induction, fecal samples were collected on three separate days during the acclimation period and examined qualitatively for the presence of Giardia cysts. All three samples must be negative for inclusion in the study. Beginning on the fourth day after inoculation, individual fecal samples were collected daily from each dog. Samples from alternate days were subjected to qualitative analysis. After cysts were observed in at least one dog, fecal samples were collected and analyzed daily, and the numbers of Giardia cysts were counted and reported on a cysts per gram (CPG) basis. Thirty-four dogs were confirmed with patent infection. These dogs were randomized, in blocks of four-to-six dogs, into one of the six treatment groups as described in Table 12. Dogs were dosed with placebo or the sustained release formulation of metronidazole in dosages ranging from 75 mg/kg to 175 mg/kg, administered as shown below.

TABLE 12

| Tx. | | | |
|---|---|---|---|
| Group | # of dogs | Treatment | Dosage |
| 1 | 4 | S.C. injection | 175 mg/kg (35 mg/kg over 5 days) |
| 2 | 6 | of extended- | 150 mg/kg (30 mg/kg over 5 days) |
| 3 | 6 | release | 125 mg/kg (25 mg/kg over 5 days) |
| 4 | 6 | metronidazole | 100 mg/kg (20 mg/kg over 5 days) |
| 5 | 5 | | 75 mg/kg (15 mg/kg over 5 days) |
| 6 | 6 | Placebo | Volume matched to 175 mg/kg |

Each dog was treated with the Investigational Veterinary Product (IVP) starting on the third day after the infection criterion was achieved (Day 0). The standard infection criterion was defined as three consecutive days (Days −3, −2, and −1) with fecal cyst counts >1,000 CPG. Daily fecal cyst counts (FCCs) for each dog continued from the time that the dog achieved the infection criterion until study exit.

### Data Processing

If the FCC of a given sample was below detection, it was replaced by half of the detection limit (67 CPG) for visual representation, data summary and statistical analysis purposes. The situation of zero count was reported as non-detect (ND) in report tables and figures. A dog was considered a treatment success when the dog had the geometric mean FCC from Day 5 to 7 less than 10% of that from Day −3 to −1.

### Statistical Analysis

The log 10-transformed geometric mean of FCC from Day 5 to 7 was analyzed using the linear mixed model with the fixed effect being treatment group. The log 10-transformed geometric mean FCC from Day −3 to −1 served as the covariate of the model. The variance of the error term was taken as heterogenous with respect to treatment group and had a lower bound of 10⁻⁴. Denote LSM(k) as the least squares mean of treatment group k, k=1, 2, 3, 4, 5, adjusted by baseline FCC. The fecal cyst count reduction (FCCR) was used to measure treatment success of treatment group k relative to placebo. It was estimated as

$$FCCR = 1 - {10}^{LSM ( TGk ) - LSM ( TG 6 )}$$

where 10^{LSM(TGk)-LSM(TG6)} represents the model-based estimate of geometric mean ratio for TGk:Placebo.

Responder analysis was also performed on the binary outcome of treatment success, at the individual animal level, using the Fisher's exact test.

All tests was performed at the 0.10 significance level. Comparisons between two treatment groups were carried out using 2-sided tests. No multiplicity adjustment was performed. Statistical analyses were carried out using the Statistical Analysis Software (SAS version 9.4; Cary, NC) MIXED and FREQ procedures.

### Results

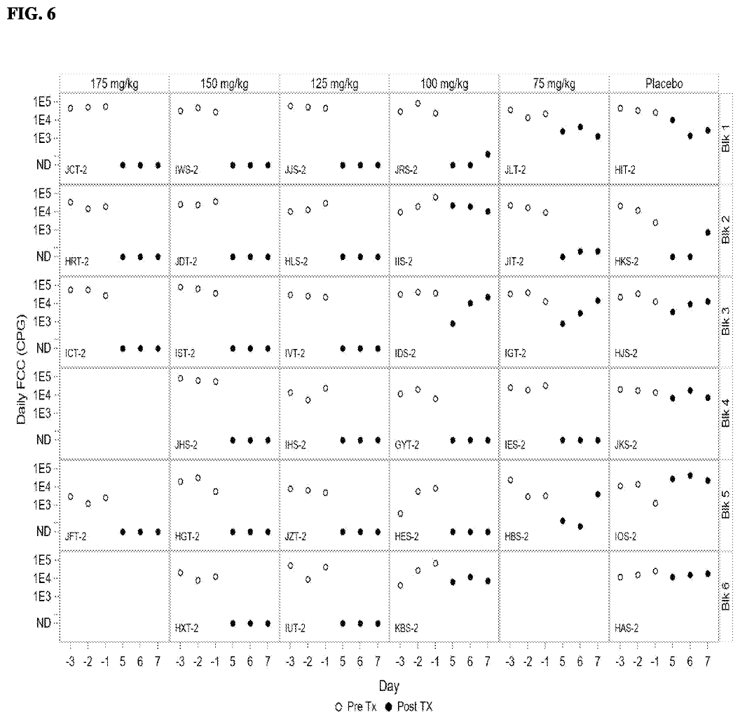

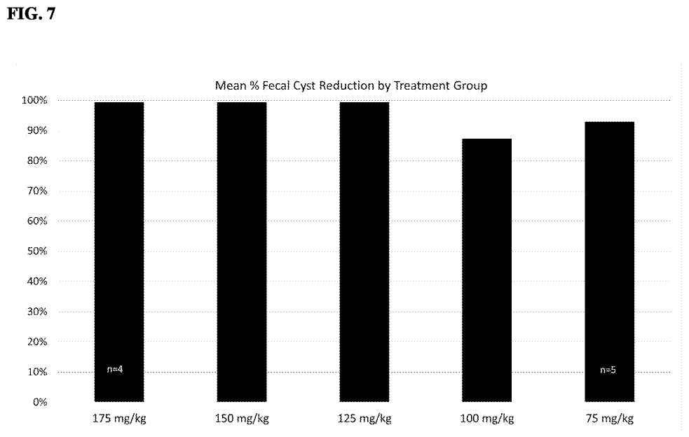

Summary statistics of study endpoints are provided in Table 13 and 14, and the data are plotted in FIG. 6. Tables 15 and 16 report the statistical analysis results. Each of the dosages provided fecal cyst reduction in the treated dogs (FIG. 7).

TABLE 13

| | | FCC Geometric Mean From | | | | FCC Geometric Mean From | | | |
|---|---|---|---|---|---|---|---|---|---|
| | | Day −3 to −1 (CPG) | | | | Day 5 to 7 (CPG) | | | |
| Tx | N | Mean | Median | Min | Max | Mean | Median | Min | Max |
| 175 mg/kg | 4 | 3.02E+04 | 3.31E+04 | 2.08E+03 | 5.23E+04 | ND | ND | ND | ND |
| 150 mg/kg | 6 | 3.67E+04 | 3.19E+04 | 1.30E+04 | 6.97E+04 | ND | ND | ND | ND |
| 125 mg/kg | 6 | 2.34E+04 | 2.12E+04 | 6.14E+03 | 5.24E+04 | ND | ND | ND | ND |
| 100 mg/kg | 6 | 2.25E+04 | 2.17E+04 | 2.52E+03 | 3.96E+04 | 5.18E+03 | 2.92E+03 | ND | 1.69E+04 |
| 75 mg/kg | 5 | 1.93E+04 | 2.32E+04 | 6.01E+03 | 2.61E+04 | 1.20E+03 | 3.30E+02 | ND | 3.25E+03 |
| Placebo | 6 | 1.77E+04 | 1.75E+04 | 5.96E+03 | 3.51E+04 | 1.10E+04 | 8.62E+03 | 9.29E+01 | 2.99E+04 |

TABLE 14

| | 1 - FCC Geometric Mean Ratio of Day 5 | | | | | |
|---|---|---|---|---|---|---|
| | to 7 vs. Day −3 to −1 | | | | | # of |
| Tx | N | Mean | Median | Min | Max | Successes |
| 175 mg/kg | 4 | 99.53% | 99.89% | 98.42% | 99.94% | 4 |
| 150 mg/kg | 6 | 99.87% | 99.90% | 99.75% | 99.95% | 6 |
| 125 mg/kg | 6 | 99.78% | 99.83% | 99.46% | 99.94% | 6 |
| 100 mg/kg | 6 | 78.21% | 91.65% | 27.66% | 99.87% | 3 |
| 75 mg/kg | 5 | 94.30% | 94.51% | 87.54% | 99.87% | 3 |
| Placebo | 6 | −15.25% | 55.49% | −401.9% | 98.92% | 2 |

TABLE 15

| | Back-Transformed LSM | | |
|---|---|---|---|
| | of Three-Day FCC | | P-value for Rejecting the |
| Tx | Geometric Mean (CPG) | FCCR | Null Hypothesis† |
| 175 mg/kg | ND | 99.30% | 0.002 |
| 150 mg/kg | ND | 99.30% | 0.002 |
| 125 mg/kg | ND | 99.30% | 0.002 |
| 100 mg/kg | 6.00E+02 | 87.23% | 0.203 |
| 75 mg/kg | 3.38E+02 | 92.81% | 0.068 |
| Placebo | 4.70E+03 | — | — |

TABLE 16

| | # of Successes/# of | P-value for Rejecting |
|---|---|---|
| Tx | Dogs | the Null Hypothesis† |
| 175 mg/kg | 4/4 | 0.076 |
| 150 mg/kg | 6/6 | 0.061 |
| 125 mg/kg | 6/6 | 0.061 |
| 100 mg/kg | 3/6 | 1.000 |
| 75 mg/kg | 3/5 | 0.567 |
| Placebo | 2/6 | — |

### Example 8: Additional Pharmacokinetics Studies

Additional formulations (see Table 17) of metronidazole were administered by a single subcutaneous administration at 125 mg/kg dose to beagle dogs and the pharmacokinetics were analyzed.

TABLE 17

| | | Particle Size (microns) | | |
|---|---|---|---|---|
| Formulation | % Drug load (w/w) | D₁₀ | D₅₀ | D₉₀ |
| 8A | 38.9 (80:20 SAIB:EA) | 30.5 | 109 | 221 |
| 8B | 43.3 (75:25 SAIB:EA) | 30.6 | 101 | 200 |
| 8C | 36.5 (75:25 SAIB:EA) | 4.8 | 20.8 | 38.0 |
| 8D | 38.3 (75:25 SAIB:EA) | 8.6 | 33.2 | 145 |
| 8E | 37.1 (75:25 SAIB:EA) | 4.9 | 21.3 | 39.3 |
| 8F | 39.5 (75:25 SAIB:EA) | 34.3 | 99.6 | 187 |

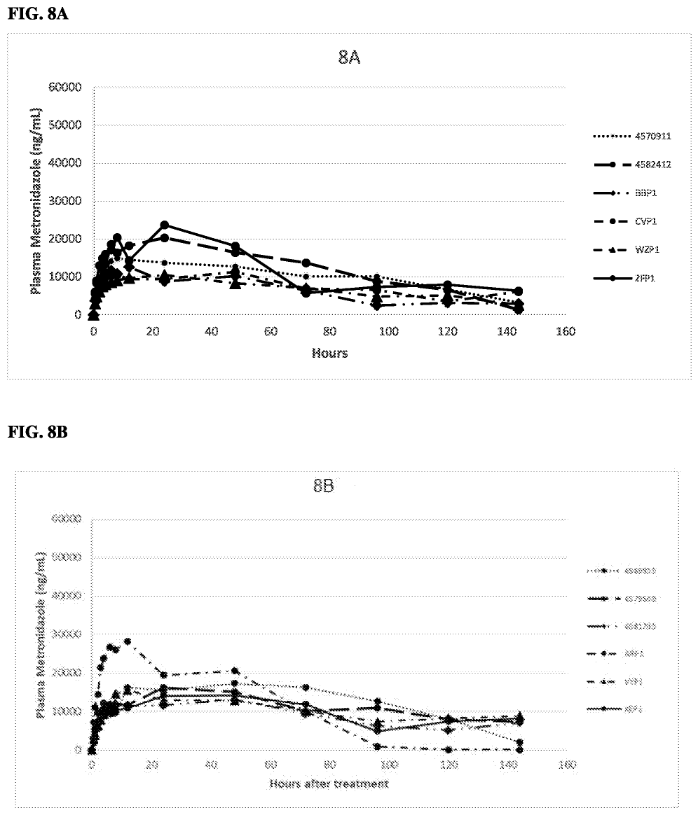

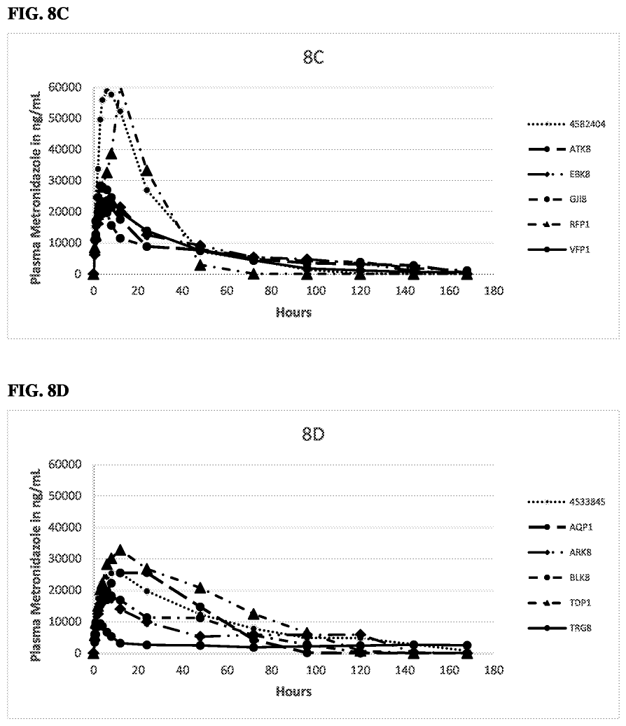

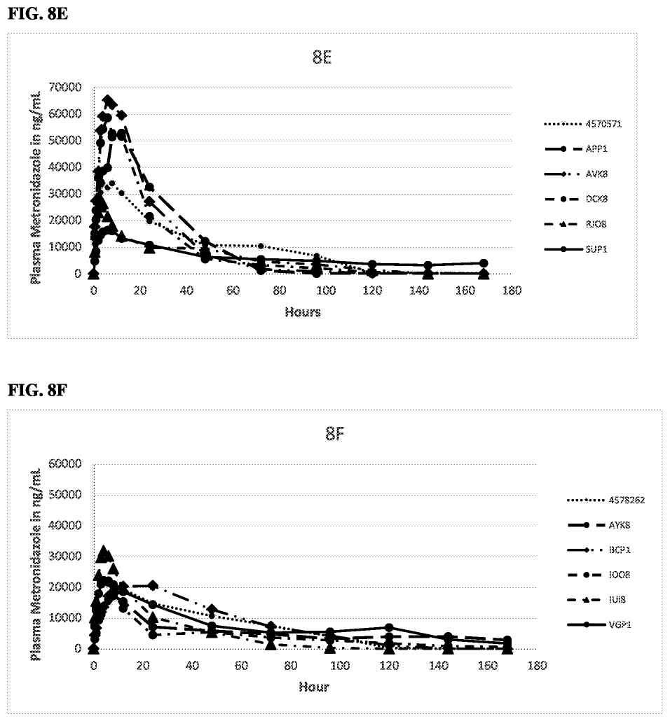

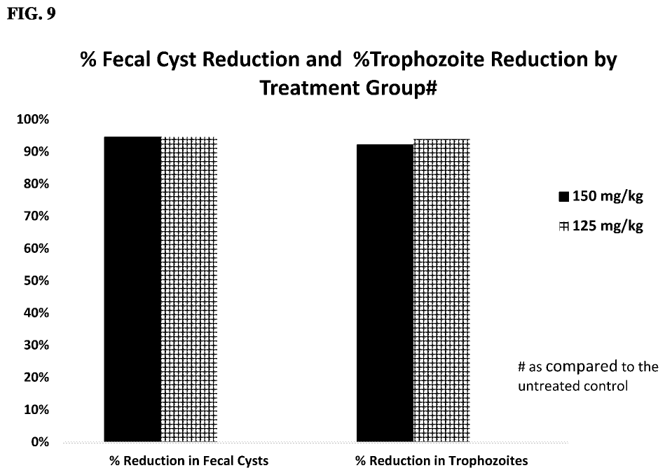

Graphs of individual animal concentration profiles for formulations 8A-8F are shown in FIGS. 8A-8F, respectively.

### Example 9: A Target Animal Pilot Dose-Finding and Preliminary Safety Study of an Extended-Release Formulation of Metronidazole at Two Dosages Administered Subcutaneously for the Treatment of Giardia in Dogs

### Objective

The major objective of the study was to determine the safety and efficacy of two dosage regimens of a metronidazole extended-release formulation; 125 mg·kg and 150 mg/kg administered in a single subcutaneous injection. Both of these dosage regimens were shown to be successful for the treatment of giardiasis in a prior study.

### Study Design

Dogs were assigned to treatment in replicates of three dogs each as the dogs met the infection criterion (IC) for assignment to treatment. The IC was defined as three consecutive days with fecal cyst counts >1000 cysts per gram (CPG) for an individual dog. Beginning on Day 7 after inoculation, daily fecal samples were examined qualitatively for the presence of cysts. After at least one dog met the IC, fecal samples were examined daily, and the numbers of Giardia cysts were counted by a standardized method using immunofluorescent microscopy and reported on a cysts per gram (CPG) basis. Within each replicate of three enrolled dogs, the dogs were ranked first according to their chronology in achieving the IC, and then in decreasing magnitude of geometric mean cyst count for randomization. Dogs were treated approximately three days after achieving the IC by intrascapular subcutaneous injection of the IVP or a saline control according to their assigned treatment group. Only 24 dogs ultimately met the infection criterion, and the table below reflects the actual assigned “n” for each treatment group:

| Tx. | | | |
|---|---|---|---|
| group | # of dogs* | Treatment | Dosage |
| 1 | 8 | Metronidazole extended- | 150 mg/kg |
| | | release sterile injectable | (30 mg/kg over 5 days) |
| | | suspension | |
| 2 | 8 | Metronidazole extended- | 125 mg/kg |
| | | release sterile injectable | (25 mg/kg over 5 days) |
| | | suspension | |
| 3 | 8 | Sterile Saline | Volume matched to 150 |
| | | | mg/kg |

*original target enrollment was 10 per group, but only 8 replicates achieved infection criterion.

General health observations were conducted daily, and fecal consistency scores were assigned at least once daily for each enrolled animal. On the day of treatment and every day thereafter until the dog completed the study, injection site reactions were monitored for heat, redness, pain, and swelling. At weekly intervals, body weights were measured, and body condition scores were assigned by a trained observer.

On days 5-7 post-treatment, fecal cyst counts (FCCs) were enumerated by quantitative Merifluor™, and within three days after the last FCC, the dogs were sacrificed, and representative intestinal segments were collected for intestinal trophozoite counts (ITC). Treatment success was determined by the following criteria:
1. A statistically significant reduction (P<0.10) in geometric mean post-treatment FCC and ITC counts in the treated groups as compared to the contemporaneous geometric means of the control group, and,
2. The point estimate of treated-vs-control-group geometric mean ratio for both FCC and ITC must be no greater than 10% (i.e., at least 90% reduction in geometric mean).

All tests were performed at the 0.05 significance level. Comparisons between two treatment groups were carried out using 2-sided tests. Because the study did not enroll the 10 dogs/group needed for 80% power to show a difference between groups on the cyst counts.

### Results

Both the 150 mg/kg, and the 125 mg/kg treatment groups demonstrated reduction in FCC of 95% as compared to the negative control after treatment. This comparative reduction was statistically significant with a P value of 0.007 for both groups (alpha=0.05).

| | | | Efficacy | P-value for |
|---|---|---|---|---|
| | | Geometric | Relative to | Testing if |
| Endpoint | Tx | Mean | Saline | Efficacy = 0† |
| FCC | Metronidazole 150 mg/kg | 3.50E+01 | 94.53% | 0.007 |
| | Metronidazole 125 mg/kg | 3.50E+01 | 94.53% | 0.007 |
| | Saline | 6.40E+02 | — | — |
| ITC | Metronidazole 150 mg/kg | 4.96E+02 | 92.08% | 0.104 |
| | Metronidazole 125 mg/kg | 3.82E+02 | 93.91% | 0.079 |
| | Saline | 6.27E+03 | — | — |

Intestinal trophozoite counts in the 150 mg/kg and 125 mg/kg were reduced by 92% and 94% respectively. Due to the lower than anticipated enrollment, the study was not powered to determine significance at alpha=0.05, however, the reduction in intestinal trophozoites in the 125 mg/kg treatment group as compared to the control was significant at alpha=0.10 (P=0.079).

### Safety

No dogs demonstrated any negative general health observations that could be attributed to treatment at any time during the study. Ten dogs developed mild swelling at the injection site around the drug depot; six of these dogs were in the 150 mg/kg dose group and four were in the 125 mg/kg dose group. Due to the nature of the product, a palpable depot in the area is to be expected, and the injection depot was not considered when swelling was reported, but any measurements necessarily included the depot. The swellings observed were most often soft with no margins and could not be accurately measured. The depot could be measured in only four dogs, and in only one dog did the affected area exceed the volume of the depot. In all but one dog in the 125 mg/kg group, the swelling was intermittent and/or resolved itself before the 8th day after injection. Aside from the three incidences of pain on palpation affecting two dogs, the swellings did not appear to cause any discomfort to the dogs.

### Conclusions

The extended-release suspension of metronidazole was effective for the treatment of induced infections of Giardia duodenalis when administered in a single treatment of 125 mg/kg or 150 mg/kg (see FIG. 9). The reduction of fecal cysts was significant at an alpha of 0.05, and the reduction in intestinal trophozoites was significant at an alpha of 0.10 for the 125 mg/kg treatment, while the 150 mg/kg dose just missed significance with P=0.104, however, the study was underpowered to determine efficacy based on trophozoite reduction. The treatment was overall well tolerated, except for mild swelling surrounding the injection site in ten of the sixteen animals treated with the drug. With two isolated exceptions this swelling was largely painless, and in all but one case, resolved within 7 days after treatment.

### INCORPORATION BY REFERENCE

All U.S. patents and published U.S. and PCT patent applications mentioned in the description above are incorporated by reference herein in their entirety.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

1. A pharmaceutical composition, comprising
(i) metronidazole or a pharmaceutically acceptable salt thereof;
(ii) sucrose acetate isobutyrate (SAIB); and
(iii) ethyl acetate.

2. The pharmaceutical composition of claim 1, comprising about 20% to about 50% w/w metronidazole or a pharmaceutically acceptable salt thereof.

3. The pharmaceutical composition of claim 1, comprising about 20% to about 45% w/w metronidazole or a pharmaceutically acceptable salt thereof.

4. The pharmaceutical composition of claim 1, comprising about 30% to about 45% w/w metronidazole or a pharmaceutically acceptable salt thereof.

5. (canceled)

6. The pharmaceutical composition of claim 4, comprising about 30% to about 40% w/w metronidazole or a pharmaceutically acceptable salt thereof.

7. The pharmaceutical composition of claim 4, comprising about 34% to about 36% w/w, about 36% to about 44% w/w, or about 38% to about 42% w/w metronidazole or a pharmaceutically acceptable salt thereof.

8. (canceled)

9. The pharmaceutical composition of claim 1, comprising about 30% to about 70% w/w SAIB.

10. The pharmaceutical composition of claim 9, comprising about 40% to about 60% w/w SAIB.

11. The pharmaceutical composition of claim 10, comprising about 50% to about 60% w/w SAIB.

12. (canceled)

13. The pharmaceutical composition of claim 1, comprising about 5% to about 20% w/w ethyl acetate.

14. (canceled)

15. (canceled)

16. The pharmaceutical composition of claim 1, further comprising (iv) an excipient in an amount of about 5% to about 15% w/w excipient.

17. The pharmaceutical composition of claim 16, wherein the excipient comprises medium-chain triglycerides.

18. (canceled)

19. (canceled)

20. (canceled)

21. (canceled)

22. The pharmaceutical composition of claim 1, wherein the weight ratio of SAIB to ethyl acetate is about 60:40 to about 90:10.

23. The pharmaceutical composition of claim 22, wherein the weight ratio of SAIB to ethyl acetate is about 70:30 to about 85:15 or about 75:25 to about 80:20.

24. (canceled)

25. (canceled)

26. (canceled)

27. The pharmaceutical composition of claim 1, wherein the metronidazole or salt thereof has a particle size distribution (D₉₀) range of about 30 μm to about 300 μm.

28. (canceled)

29. (canceled)

30. (canceled)

31. The pharmaceutical composition of claim 1, wherein the metronidazole or salt thereof has a particle size distribution (D₅₀) range of about 10 μm to about 120 μm.

32. (canceled)

33. (canceled)

34. (canceled)

35. The pharmaceutical composition of claim 1, comprising.
(i) about 30% to about 42% w/w metronidazole or a pharmaceutically acceptable salt thereof:
(ii) about 48% to about 52.5% w/w SAIB; and
(iii) about 16% to about 17.5% w/w ethyl acetate.

36. The pharmaceutical composition of claim 1, wherein the pharmaceutical composition is an extended-release composition.

37. A method of delivering metronidazole or a pharmaceutically acceptable salt thereof, comprising administering to a non-human subject in need thereof an effective amount of the pharmaceutical composition of claim 1, thereby delivering the metronidazole or a pharmaceutically acceptable salt thereof.

38.-43. (canceled)

44. A method of treating or preventing a protozoal or bacterial infection or disease, comprising administering to a non-human subject in need thereof a therapeutically effective amount of the pharmaceutical composition of claim 1, thereby treating or preventing the protozoal or bacterial infection or disease.

45.-72. (canceled)
